(19) United States
(12) Patent Application Publication
(10) Pub. No.: US 2016/0317108 A1
(43) Pub. Date: Nov. 3, 2016

(54) DENTAL APPLIANCE WITH A REGISTRATION FIDUCIAL

(71) Applicant: Claronav Inc., North York (CA)

(72) Inventors: Doron Dekel, Toronto (CA); Arish Qazi, Milton (CA)

(73) Assignee: CLARONAV INC.

(21) Appl. No.: 15/209,945

(22) Filed: Jul. 14, 2016

Related U.S. Application Data
(63) Continuation of application No. PCT/CA2015/050025, filed on Jan. 15, 2015.
(60) Provisional application No. 61/927,839, filed on Jan. 15, 2014.

(51) Int. Cl. A61B 6/14; A61B 6/00; A61B 6/03; A61C 9/00; A61B 6/12

(52) U.S. Cl. CPC A61B 6/145; A61C 9/0046; A61C 9/0006; A61B 6/12; A61B 6/032; A61B 6/5229

(57) ABSTRACT

An apparatus and system are provided for registering a human jaw with a scanned image of the human jaw. The apparatus is repeatably attachable to the jaw and comprises a fiducial body. The surface of the fiducial body includes a plurality of feature regions and defines a fiducial plane. The fiducial body has a curved profile shaped to follow the curve of the jaw when the appliance is attached to the jaw. When the jaw is scanned with the appliance attached thereto, a representation of at least a portion of the surface of the fiducial body is identifiable as a boundary in the scanned image. A unique coordinate mapping between the fiducial body and the scanned image is determinable from the fiducial plane and the plurality of geometrical characteristics at least one and fewer than three feature regions. The system comprises a scanner and a data processor.

### CROSS-REFERENCE TO RELATED APPLICATION

This application is a continuation of PCT Patent Application No. PCT/CA2015/050025 filed on Jan. 15, 2015, entitled “DENTAL APPLIANCE WITH A REGISTRATION FIDUCIAL” which claims priority from the U.S. Provisional Patent Application No. 61/927,839, filed Jan. 15, 2014, entitled “REPEATABLE JAW ATTACHMENT”. The entirety of the contents of PCT Patent Application No. PCT/CA2015/050025 and U.S. Provisional Patent Application No. 61/927,839 are herein incorporated by reference.

### FIELD

The described embodiments relate to the field of medicine, in particular, the field of dental navigation systems.

### BACKGROUND

PCT Application No. PCT/CA2011/001294 (“PCT1294” herein), incorporated herein by reference in its entirety, teaches the use of an apparatus conforming to the surface of an anatomical region to attach one or more rigid bodies to the anatomical region at a repeatable position relative to the anatomical region. Such repeatable fixation may enable geometrical mapping of anatomical regions across clinical procedures performed at different times. For example, during a surgical procedure, an anatomical region may be dynamically registered with an image of the anatomical region to provide guidance to the surgeon. As well, the range and patterns of motions of anatomical regions may be measured and compared. Furthermore, the anatomical region may be repositioned relative to an instrument for various forms of treatment.

While methods of repeatedly attaching a rigid structure to the human jaw using the rigid surfaces of the teeth are known, such methods are typically inconvenient because they require multiple patient visits. During a first patient visit, personnel at a medical office create a model of the surface of the human jaw by taking a physical or a digital (scanner) impression. Next, trained technicians at a separate laboratory facility prepare a special appliance, a “stent”, based on the model. At a subsequent patient visit, personnel at the medical office evaluate the fit of the appliance.

In some cases, these known methods may not produce satisfactory appliances. The appliance may be too loose to provide stable, rigid, attachment (“clicking” to place). The appliance may also be too tight, causing difficulty in inserting and removing the attachment without risking damage to either the anatomical region or the appliance.

Commonly in current surgical navigation systems, registration between a human jaw and a scanned image of the human jaw is done by identifying the locations of corresponding point target locations in both the image and the real-world (position-tracked) coordinate spaces. Often this is achieved by placing high density spherical objects of known diameter (“fiducial markers”) in proximity to the anatomical region both when the patient is scanned to produce the image and prior to the start of the surgical procedure. These fiducial targets may be placed individually at locations chosen by a user of the navigation system. Alternatively, multiple fiducial markers may be pre-mounted on a carrier and placed together as a group.

In such approach, a minimum of 3 corresponding target locations is necessary to enable computing a unique rigid 3D registration mapping. Sometimes less than 3 fiducial markers are identified in the image, for example due to some markers falling outside the image's limited field of view, or due to image corruption or noise obscuring the markers' appearance. In such a case, reliable registration is not possible and navigation needs to be abandoned. When a single fiducial body is used, as disclosed in PCT1294, a large portion of the fiducial body needs to be identified in the image for a successful registration to take place. Registration is then not possible, or is inaccurate, when only a small portion of the fiducial body appears in the image, or when the appearance of the fiducial in the image is degraded in parts.

### SUMMARY

In accordance with an embodiment, there is provided an apparatus for registering a human jaw with a scanned volumetric image of the human jaw. The apparatus can include an appliance repeatably attachable to the human jaw, the appliance having a rigid fiducial body and constructed such that when the appliance is attached to the human jaw the rigid fiducial body is in a fixed spatial relationship with the human jaw. A surface of the rigid fiducial body can include a side surface and an opposite side surface, the opposite side surface spaced apart from the side surface throughout the rigid fiducial body and at least one of the side surface and the opposite side surface can define a fiducial plane. The surface of the rigid fiducial body can also include a plurality of feature regions where each feature region defines a feature point location and a plurality of geometrical characteristics associated with that feature point location. The rigid fiducial body can have a curved profile such that a projection of the curved profile onto the fiducial plane is generally curved; the curved profile can be shaped to follow a curve of the human jaw when the appliance is attached to the human jaw. The rigid fiducial body can be formed such that when the human jaw is tomographically scanned with the appliance attached thereto, a representation of at least a portion of the surface of the rigid fiducial body can be identified as a boundary in the scanned volumetric image. When the portion of the surface identifiable as the boundary in the scanned volumetric image includes two of the feature regions, a unique coordinate mapping between the rigid fiducial body and the scanned volumetric image can be determined from the fiducial plane and the plurality of geometrical characteristics for each of the two feature regions. The plurality of geometrical characteristics for each of the two feature regions and the fiducial plane are determinable from the boundary in the scanned volumetric image.

In accordance with another embodiment, there is provided an apparatus for registering a human jaw with a scanned volumetric image of the human jaw. The apparatus can include an appliance repeatably attachable to the human jaw, where the appliance includes a rigid fiducial body and is constructed such that when the appliance is attached to the human jaw the rigid fiducial body is in a fixed spatial relationship with the human jaw. A surface of the rigid fiducial body can include a plurality of feature regions, where each feature region defines a feature point location and a plurality of geometrical characteristics associated with that feature point location. The surface of the rigid fiducial body can also include a side surface and an opposite side surface, the opposite side surface spaced apart from the side surface throughout the rigid fiducial body, at least one of the side surface and the opposite side surface defining a fiducial plane. The rigid fiducial body can have a curved profile such that a projection of the curved profile onto the fiducial plane is generally curved, the curved profile can be shaped to follow a curve of the human jaw when the appliance is attached to the human jaw. The rigid fiducial body can be formed such that when the human jaw is tomographically scanned with the appliance attached thereto, a representation of at least a portion of the surface of the rigid fiducial body can be identified as a boundary in the scanned volumetric image. When the portion of the surface identifiable as the boundary in the scanned volumetric image includes one of the feature regions, a unique coordinate mapping between the rigid fiducial body and the scanned volumetric image can be determined from the fiducial plane and the plurality of geometrical characteristics for that feature region. The fiducial plane and the plurality of geometrical characteristics of that feature region are determinable from the boundary in the scanned volumetric image.

In some cases, for each feature region in the plurality of feature regions the plurality of geometrical characteristics and the fiducial plane can define a feature point location relative to the rigid fiducial body for that feature region.

In some cases, the side surface and the opposite side surface can together define the fiducial plane and be substantially symmetrical about the fiducial plane. In some cases, the side surface and the opposite side surface can define a fiducial plane substantially parallel to each of the side surface and the opposite side surface.

In some cases, the side surface can be substantially planar such that the side surface defines the fiducial plane.

In some cases, the curved profile can be substantially arch shaped. In some cases, the curved profile can be shaped to keep a substantially uniform distance between the surface of the rigid fiducial body and a portion of an external surface of an average human jaw. In some cases, the curved profile can be shaped to keep a substantially uniform distance between the surface of the rigid fiducial body and a portion of a buccal surface of an average human jaw.

In some cases, a distance between the side surface and the opposite side surface can define a thickness of the rigid fiducial body. For a first feature region of the plurality of feature regions, the thickness of the rigid fiducial body in that first feature region can be different from the thickness of the rigid fiducial body in a second feature region, the thickness of the rigid fiducial body in the first feature region can be one of the geometrical characteristics of the first feature region, and the thickness of the rigid fiducial body in the second feature region can be one of the geometrical characteristics of the second feature region.

In some cases, the feature point location of each feature region can be one of a center point and a vertex point.

In some cases, when the feature point location of at least one of the feature regions is a vertex point, for each feature region where the feature point location is a vertex point the surface of the rigid fiducial body can include a first edge surface portion extending from the side surface to the opposite side surface in that feature region, and a second edge surface portion extending from the side surface to the opposite side surface in that feature region. An intersection of the first edge surface portion and the fiducial plane within the feature region can define a first edge intersecting line that is substantially straight, an intersection of the second edge surface portion and the fiducial plane within the feature region can define a second edge intersecting line that is substantially straight. The vertex point can be formed by an intersection of the first edge intersecting line and the second edge intersecting line at the fiducial plane. The vertex point can have an opening angle formed by the intersection of the first edge intersecting line and the second edge intersecting line at the vertex point, the opening angle having an opening angle magnitude. The geometrical characteristics of that feature region can include the opening angle magnitude.

In some cases, for a first feature region where the feature point location is a vertex point, the opening angle magnitude can be distinct from the opening angle magnitude of a second feature region where the feature point location is a vertex point, the opening angle magnitude of the first feature region can be one of the geometrical characteristics of the first feature region and the opening angle magnitude of the second feature region can be one of the geometrical characteristics of the second feature region.

In some cases, for each feature region where the feature point location is a vertex point, that feature region can define an opening angle bisecting direction in a direction originating from the vertex point and bisecting the opening angle. The geometrical characteristics of that feature region can include the opening angle bisecting direction.

In some cases, where the feature point location of at least one of the feature regions is a center point, for each feature region where the feature point location is a center point the surface of the rigid fiducial body can include a first edge surface portion extending from the side surface to the opposite side surface in that feature region, the first edge surface portion can have a circular segment profile such that a projection of the circular segment profile onto the fiducial plane is a circular segment, the circular segment profile having a circle segment radius. An intersection of the fiducial plane and a center of the circular segment can define the center point and the geometrical characteristics of that feature region can include the circle segment radius.

In some cases, for each feature region where the feature point location is a center point the circle segment radius of that feature region is different from the circle segment radius of any other feature region comprising a center point.

In some cases, for each feature region where the feature point location is a center point the center point can have an opening angle defined by an angle subtended by the first edge surface portion through the center point, the opening angle having an opening angle magnitude and the geometrical characteristics of that feature region can include the opening angle magnitude.

In some cases, for each feature region where the feature point location is a center point a combination of the circle segment radius and the opening angle magnitude of that feature region is different from a combination of circle segment radius and opening angle magnitude of any other feature region comprising a center point.

In some cases, for each feature region where the feature point location is a center point the feature region can define an opening angle bisecting direction in a direction originating from the center point and bisecting the opening angle and the geometrical characteristics of that feature region can include the opening angle bisecting direction.

In some cases, for each feature region the surface of the rigid fiducial body comprises at least one edge surface portion extending from the side surface to the opposite side surface that is asymmetrical about the fiducial plane within that feature region.

In some cases, the rigid fiducial body includes a plurality of feature region pairs, with each feature region pair including a first feature region and a second feature region, where each feature region pair has a distinct combination of geometrical characteristics. When the portion of the surface identifiable as the boundary in the scanned volumetric image includes any feature region pair, the unique coordinate mapping between the rigid fiducial body and the scanned volumetric image can be determined from the fiducial plane and the combination of geometrical characteristics for that feature region pair.

In some cases, the portion of the surface identifiable as the boundary in the scanned volumetric image can include a first sub-portion including the first feature region of the feature region pair and a second sub-portion including the second feature region of the feature region pair where the first sub-portion and the second sub-portion are disjoint in the scanned volumetric image.

In some cases, the combination of geometrical characteristics for any feature region pair can include: the first opening angle magnitude of the opening angle of the first feature region, the second opening angle magnitude of the opening angle of the second feature region; the magnitude of a first pair vector from the feature point location of the first feature region to the feature point location of the second feature region, a first vector bisector angle defined by the angle between the first pair vector and the opening angle bisecting vector in the direction originating from the feature point location of the first feature region and bisecting the opening angle of the first feature region, and a second vector bisector angle defined by the angle between the first pair vector and the opening angle bisecting vector in the direction originating from the feature point location of the second feature region and bisecting the opening angle of the second feature region.

In some cases, the geometrical characteristics of each feature region can include a unique combination of the opening angle magnitude of an opening angle of that feature region, the distance from a feature point location of that feature region to a feature point location of a first adjacent feature region, and the distance from the feature point location of that feature region to a feature point location of a second adjacent feature region.

In some cases, the appliance can be configured to be moldable to an appliance geometry that mates with a surface geometry of at least a portion of the human jaw, such that when mated with the human jaw, the appliance resists displacement relative to the human jaw, the appliance can be further configured to be hardenable to remain rigid and resist deformation once molded to the appliance geometry.

In some cases, a portion of the appliance is rigid below 45° C., the portion becomes moldable when heated to a transition temperature greater than 45° C. and less than 100° C., the portion being subsequently hardenable when cooled below 45° C.

In accordance with an embodiment, there is provided a system for registering a human jaw with a scanned volumetric image of the human jaw. The system can include a tomographic scanner for conducting a scan of the human jaw with an apparatus attached thereto, the apparatus can include a rigid fiducial body and be constructed such that the rigid fiducial body is maintained in a fixed spatial relationship with the human jaw when the apparatus is attached thereto, the rigid fiducial body can be formed such that when the human jaw is scanned with the apparatus attached thereto, a representation of at least a portion of the surface of the rigid fiducial body is identifiable as a boundary in the scanned volumetric image, the scanner can be configured to provide a representation of the human jaw and the portion of a surface of the rigid fiducial body distinguishable as the boundary in the scanned volumetric image, where the portion of the rigid fiducial body includes at least one feature region, where each feature region has a plurality of geometrical characteristics. The system can also include a data processor coupled to the tomographic scanner. The data processor can be configured to receive the scanned volumetric image from the tomographic scanner, identify a fiducial plane in the scanned volumetric image, and identify a first feature region representation and a first feature point location representation in the scanned volumetric image based on a plurality of geometric characteristics of the first feature region representation and the fiducial plane, the first feature region representation corresponding to a first feature region in the portion of the surface of the rigid fiducial body. The data processor can be further configured to determine a unique coordinate mapping between the scanned volumetric image and the rigid fiducial body using the identified fiducial plane and the geometrical characteristics of the identified first feature region.

In accordance with another embodiment, there is provided a system for registering a human jaw with a scanned volumetric image of the human jaw. The system can include a tomographic scanner for conducting a scan of the human jaw with an apparatus attached thereto, the apparatus can include a rigid fiducial body and be constructed such that the rigid fiducial body is maintained in a fixed spatial relationship with the human jaw when the apparatus is attached thereto, the rigid fiducial body can be formed such that when the human jaw is scanned with the apparatus attached thereto, a representation of at least a portion of the surface of the rigid fiducial body is identifiable as a boundary in the scanned volumetric image, the scanner can be configured to provide a representation of the human jaw and the portion of a surface of the rigid fiducial body distinguishable as the boundary in the scanned volumetric image, where the portion of the rigid fiducial body comprising at least two feature regions, and each feature region has a plurality of geometrical characteristics. The system can also include a data processor coupled to the tomographic scanner.

The data processor can be configured to receive the scanned volumetric image from the tomographic scanner, identify a fiducial plane in the scanned volumetric image, and identify a first feature region representation and a first feature point location representation in the scanned volumetric image based on a plurality of geometric characteristics of the first feature region representation and the fiducial plane, the first feature region representation corresponding to a first feature region in the portion of the surface of the rigid fiducial body. The data processor can also be configured to identify a second feature region representation and a second feature point location representation in the scanned volumetric image based on a plurality of geometric characteristics of the second feature region representation and the fiducial plane, the second feature region representation corresponding to a second feature region in the portion of the surface of the rigid fiducial body, and determine a unique coordinate mapping between the scanned volumetric image and the rigid fiducial body using the identified fiducial plane and the geometrical characteristics of the identified first feature region and the identified second feature region.

In some cases, the system can also include the apparatus, wherein the apparatus is defined herein.

### BRIEF DESCRIPTION OF THE DRAWINGS

Several embodiments will now be described in detail with reference to the drawings, in which:

FIG. 1A illustrates a top view of an example embodiment of an apparatus for registering a human jaw with a scanned volumetric image of the human jaw;

FIG. 1B illustrates a bottom view of the apparatus shown in FIG. 1A;

FIG. 1C illustrates a cross-sectional view of the apparatus along the section A-B shown in FIG. 1A;

FIG. 1D illustrates a cross-sectional view of the apparatus along the section C-D shown in FIG. 1A;

FIG. 1E illustrates a cross-sectional view of the apparatus along the section E-F shown in FIG. 1A;

FIG. 1F illustrates a perspective view of an example embodiments of a rigid fiducial body of the apparatus shown in FIG. 1A;

FIG. 2A illustrates a top view of an example embodiment of a rigid fiducial body of the apparatus shown in FIG. 1A;

FIG. 2B illustrates a top view of a tag connection region of the apparatus shown in FIG. 1A with a tag attached thereto;

FIG. 3A illustrates a cross-sectional view of an example embodiment of a rigid fiducial body and a fiducial plane defined by the rigid fiducial body;

FIG. 3B illustrates a cross-sectional view of another example embodiment of a rigid fiducial body comprising a side surface and an opposite side surface and a fiducial plane defined by the rigid fiducial body;

FIG. 3C illustrates a cross-sectional view of another example embodiment of a rigid fiducial body comprising a side surface and an opposite side surface and a fiducial plane defined by the rigid fiducial body;

FIG. 3D illustrates a cross-sectional view of another example embodiment of a rigid fiducial body comprising a side surface and an opposite side surface and a fiducial plane defined by the rigid fiducial body;

FIG. 4 illustrates a top view of an example embodiment of a rigid fiducial body comprising a corner region and a circle sector region;

FIG. 5A illustrates a perspective view of an example embodiment of a corner region of a rigid fiducial body comprising a vertex point;

FIG. 5B illustrates a top view of the example embodiment of a corner region comprising a vertex point shown in FIG. 5A;

FIG. 6A illustrates a perspective view an example embodiment of a circle sector region of a rigid fiducial body comprising a center point;

FIG. 6B illustrates a perspective view of another example embodiment of a circle sector region of a rigid fiducial body comprising a center point;

FIG. 6C illustrates a top view of the example embodiment of a circle sector region comprising a center point shown in FIG. 6A;

FIG. 6D illustrates a top view of the example embodiment of a circle sector region comprising a center point shown in FIG. 6B;

FIG. 7 illustrates a top view of an example embodiment of a feature region pair of a rigid fiducial body;

FIG. 8 illustrates a top view of an example embodiment of a feature region comprising a first feature region, a first adjacent feature region, and a second adjacent feature region;

FIG. 9 illustrates an example embodiment of a system for registering a human jaw with a scanned image of the human jaw; and

FIG. 10 is a flowchart illustrating an example embodiment of a method for operating a data processor of the system shown in FIG. 9.

The drawings, described below, are provided for purposes of illustration, and not of limitation, of the aspects and features of various examples of embodiments described herein. For simplicity and clarity of illustration, elements shown in the drawings have not necessarily been drawn to scale. The dimensions of some of the elements may be exaggerated relative to other elements for clarity. It will be appreciated that for simplicity and clarity of illustration, where considered appropriate, reference numerals may be repeated among the drawings to indicate corresponding or analogous elements or steps.

### DESCRIPTION OF EXAMPLE EMBODIMENTS

It will be appreciated that numerous specific details are set forth in order to provide a thorough understanding of the example embodiments described herein. However, it will be understood by those of ordinary skill in the art that the embodiments described herein may be practiced without these specific details. In other instances, well-known methods, procedures and components have not been described in detail so as not to obscure the embodiments described herein. Furthermore, this description and the drawings are not to be considered as limiting the scope of the embodiments described herein in any way, but rather as merely describing the implementation of the various embodiments described herein.

It should be noted that terms of degree such as “substantially”, “about” and “approximately” when used herein mean a reasonable amount of deviation of the modified term such that the end result is not significantly changed. These terms of degree should be construed as including a deviation of the modified term if this deviation would not negate the meaning of the term it modifies.

In addition, as used herein, the wording “and/or” is intended to represent an inclusive-or. That is, “X and/or Y” is intended to mean X or Y or both, for example. As a further example, “X, Y, and/or Z” is intended to mean X or Y or Z or any combination thereof.

It should be noted that the term “coupled” used herein indicates that two elements can be directly coupled to one another or coupled to one another through one or more intermediate elements.

In embodiments, aspects of methods described herein, such as method 1000 described with reference to FIG. 10 below, may be implemented in hardware or software, or a combination of both. These embodiments may be implemented in computer programs executing on programmable computers, each computer including at least one processor, a data storage system (including volatile memory or non-volatile memory or other data storage elements or a combination thereof), and at least one communication interface. For example and without limitation, the programmable computer (referred to below as data processor) may be a server, network appliance, embedded device, computer expansion module, a personal computer, laptop, personal data assistant, cellular telephone, smart-phone device, tablet computer, a wireless device or any other computing device capable of being configured to carry out the methods described herein.

In some embodiments, the communication interface may be a network communication interface. In embodiments in which elements are combined, the communication interface may be a software communication interface, such as those for inter-process communication (IPC). In still other embodiments, there may be a combination of communication interfaces implemented as hardware, software, and combination thereof.

Program code may be applied to input data to perform the functions described herein and to generate output information. The output information is applied to one or more output devices, in known fashion.

Each program may be implemented in a high level procedural or object oriented programming and/or scripting language, or both, to communicate with a computer system. However, the programs may be implemented in assembly or machine language, if desired. In any case, the language may be a compiled or interpreted language. Each such computer program may be stored on a storage media or a device (e.g. ROM, magnetic disk, optical disc) readable by a general or special purpose programmable computer, for configuring and operating the computer when the storage media or device is read by the computer to perform the procedures described herein. Embodiments of the system may also be considered to be implemented as a non-transitory computer-readable storage medium, configured with a computer program, where the storage medium so configured causes a computer to operate in a specific and predefined manner to perform the functions described herein.

Furthermore, aspects of methods of the described embodiments are capable of being distributed in a computer program product comprising a computer readable medium that bears computer usable instructions for one or more processors. The medium may be provided in various forms, including one or more diskettes, compact disks, tapes, chips, wireline transmissions, satellite transmissions, internet transmission or downloadings, magnetic and electronic storage media, digital and analog signals, and the like. The computer useable instructions may also be in various forms, including compiled and non-compiled code.

The various embodiments described herein generally relate to an apparatus and associated system for registering a human jaw with a scanned volumetric image of the human jaw. Generally, the apparatus comprises an appliance comprising a rigid fiducial body. The appliance may be repeatably attachable to the human jaw such that when the appliance is attached to the jaw, the rigid fiducial body is in a fixed spatial relationship with the human jaw.

The surface of the rigid fiducial body can include a plurality of feature regions. A feature region of a surface is defined herein as a surface region designed to enable extraction of geometrical characteristics related to a nearby feature point. The geometrical characteristics of each feature region, along with a fiducial plane of the rigid fiducial body, provide information sufficient to determine a position/location of the feature point (a feature point location) of that feature region within a 3D coordinate system associated with the entire rigid fiducial body. When the boundary line representing a feature region is detected in a scanned image of that fiducial body, the same geometrical characteristics can be computed from the detected boundary and used to similarly determine the position of the feature point within a 3D coordinate system associated with the entire rigid fiducial body at the time it was scanned.

In some cases, the geometrical characteristic of a feature region and the fiducial plane may define the feature point location of that feature region. For example, in some cases the geometrical characteristics of a feature region may define a feature point line (or feature point axis) and the feature point location can be defined by a point of intersection between the feature point line and the fiducial plane.

The geometrical characteristics can also provide additional information regarding the feature region such as the orientation of that feature region in space or other geometrical characteristics defined by the feature region that may help distinguish individual feature regions (e.g. scalar values such as an angle magnitude). In different embodiments, the feature point location may or may not be inside the feature region, or even on the surface of the rigid fiducial body. Examples of feature regions include a corner region, where the feature point location can be determined based on the corner's intersection vertex at the fiducial plane which is within the feature region, and a circle sector region, where the feature point location can be defined as the intersection of the fiducial plane and center point or center line of the circle sector region and which may not lie on the surface at all.

Each feature region can define a plurality of geometrical characteristics of the rigid fiducial body. For example, in some cases each feature region may be one of a corner region and a circle sector region. Generally, a corner region may include a vertex point and opening angle of the vertex while a circle sector region may include a center point, define a circular segment radius of curvature and have a circular segment profile.

The surface of the rigid fiducial body may comprise a side surface and an opposite side surface that are spaced apart throughout the rigid fiducial body. At least one of the side surface and the opposite side surface may define a fiducial plane of the rigid fiducial body. For example, in some cases the side surface and the opposite side surface may together define the fiducial plane and be symmetrical about the fiducial plane. In some cases, the side surface may be substantially planar such that the side surface defines the fiducial plane. In some cases, the rigid fiducial body may be formed as a “flat slab”, wherein each of the side surface and the opposite side surface are substantially planar and symmetrical about the fiducial plane of the rigid fiducial body.

In some cases where a feature region includes a vertex point (i.e. a corner region), that vertex point may be formed by an intersection of two edge surface portions, each extending from the side surface to the opposite side surface, at the fiducial plane. The intersection of the two edge surface portions at the vertex point may define an opening angle of the vertex point, the opening angle having an opening angle magnitude. The geometrical characteristics of that feature region may include the opening angle magnitude of the vertex point. In some cases, the opening angle magnitude for each feature region having a vertex point may be distinct from the opening angle magnitude of every other feature region having a vertex point. In some cases, the geometrical characteristics of a feature region may also include an opening angle bisection direction, the opening angle bisection direction defined as a direction origination from the vertex point and bisecting the opening angle of that feature region.

In some cases where a feature region includes a center point (i.e. a circle sector region), an edge of the rigid fiducial body may have a circular segment profile where a projection of the circular segment profile onto the fiducial plane is a circular segment having a circle segment radius. The center point can be defined by the intersection of the fiducial plane and the center of the circular segment. The geometrical characteristics of a feature region including a center point may include the circle segment radius for the circle segment in that feature region. In some cases, the circle segment radius for each feature region having a center point is different from the circle segment radius of any other feature region having a center point. In some cases, the geometrical characteristics of a feature region with a center point may include an opening angle magnitude of an opening angle of that feature region and/or an opening angle bisecting direction.

The rigid fiducial body can have a curved profile such that a projection of the curved profile onto the fiducial plane is generally curved. This curved profile can be shaped to follow a curve of a human jaw when the appliance is attached to the jaw. For example, the curved profile may be substantially arch-shaped. In some embodiments, the rigid fiducial body may have a curved profile whose curve is not entirely smooth, but retains a substantially curved shape when projected onto the fiducial plane. For example, in some cases the feature regions may include corner regions or circle section regions along the extent of the rigid fiducial body such that there are slight variations in the surface of the rigid fiducial body while retaining a substantially curved profile. In some cases, such feature regions may be provided on a jaw-facing surface of the rigid fiducial body, where the jaw-facing surface extends from the side surface to the opposite side surface throughout the extent of the rigid fiducial body, and the jaw-facing surface is proximate the jaw when the appliance is attached to the jaw.

The curved profile can be shaped to keep a substantially uniform distance from a portion of the buccal surface of an average human jaw. For example, the surface of the fiducial body can be shaped to keep a substantially uniform distance from a portion of the external surface of an average human jaw, either on the buccal (cheeks/lips-facing) side or the occlusal (biting) side. In some cases, the appliance can be shaped to hold the fiducial inside the patient's mouth, while in other cases it can be shaped to hold the fiducial outside the mouth, near the external surface of the patient's lips and/or cheeks.

Jaws of different humans may vary in shape and dimensions. However, the external side of the arch of the jaw of an adult has an average internal curvature radius of about 20 mm, and an external curvature radius of about 30 mm. Thus, a rigid fiducial body designed to be placed proximal to the external (buccal) surface of the jaw can have a curved profile with a radius of curvature somewhat larger than 30 mm, and a rigid fiducial body designed to be placed next to the occlusal side of the jaw can have a curved profile with a radius of curvature of approximately 25 mm.

In some cases, using embodiments of methods described herein may enable embodiments of the apparatus described herein to be prepared more easily, rapidly, and reliably than previous approaches. In some cases, the apparatus may comprise an appliance with a portion made of a moldable material. The appliance may be molded directly on a human jaw or an accurate facsimile model of the human jaw, e.g. a model cast from an impression of the human jaw. In some cases the appliance may include a moldable portion, sometimes referred to as a retainer sheet and a rigid fiducial body. In some cases, the rigid fiducial body may be embedded in the moldable portion of the appliance.

The resulting moldable portion may then be molded over the surface of at least a portion of the jaw (e.g. the teeth) to provide an appliance geometry that mates with the surface geometry of at least a portion of the jaw. The moldable portion can be molded to form a tight fit with the surfaces of the jaw such that when the appliance is mated to the surface of the jaw, the appliance resists deformation relative to the jaw. The moldable portion of the appliance can then be cooled to harden it. Once hardened the appliance may resist deformation relative to its molded shape.

As mentioned above, there is also provided herein a system for registering a human jaw with a scanned image of the human jaw. The system may include a scanner for conducting a scan of the human jaw with an appliance attached thereto, the appliance including a rigid fiducial body. In different cases, the appliance may include any of the various embodiments described herein of an apparatus for registering a human jaw with a scanned image of the human jaw. In some cases, the scanner may be a tomographic scanner such as an X-ray computed tomography (CT) scanner.

The system may also include a data processor coupled to the scanner. The data processor can be configured to receive a scanned volumetric image from the scanner. The data processor may be configured to identify a fiducial plane in the volumetric image and identify a first feature region representation in the scanned volumetric image corresponding to a first feature region of a portion of the surface of a rigid fiducial body. The first feature region may be identified based on a plurality of geometric characteristics of the first feature region representation. The data processor can be further configured to determine a unique coordinate mapping between the scanned volumetric image and the rigid fiducial body using the identified fiducial plane and the geometrical characteristics of the identified first feature region.

In some cases, the system may also include a database memory for storing a reference model of the rigid fiducial body. The database memory can be coupled to the data processor. In some embodiments, the system may also include any of the embodiments of the appliance described herein.

The embodiments of the apparatus and system described herein may generally be used to register a human jaw with a scanned image of the human jaw. Accordingly, the rigid fiducial body can be formed such that when the human jaw is scanned with the appliance attached thereto, a representation of at least a portion of the surface of the rigid fiducial body is identifiable as a boundary in the scanned volumetric image. For example, the rigid fiducial body can be made of a material, such as aluminum, that is easily distinguishable from the surrounding thermoplastic in CT images but does not create image artifacts that may interfere with viewing nearby patient anatomy in the CT images. In some cases, the rigid fiducial body can be shaped such that surfaces of the rigid fiducial body defining a fiducial plane and feature regions can be identifiable as a boundary in the scanned volumetric image.

As mentioned above, the surface of a rigid fiducial body may include a plurality of feature regions. When the portion of the surface of the fiducial body identifiable as a boundary in a scanned volumetric image comprises at least one of the feature regions, a unique coordinate mapping between the rigid fiducial body and the scanned volumetric image is determinable from the fiducial plane and the plurality of geometrical characteristics for the at least one feature region and fewer than three feature regions. Thus, the embodiments of a rigid fiducial body described herein do not require three feature regions to be identified in order to provide an accurate registration with an image of the human jaw, although the unique coordinate mapping can still be determined if more than two feature regions are identified in the scanned image. In some cases, the unique coordinate mapping between the rigid fiducial body and the scanned volumetric image is determinable from the fiducial plane and the plurality of geometrical characteristics for one feature region. The fiducial plane and the plurality of geometrical characteristics of the at least one feature region can also be determined from the boundary in the scanned volumetric image.

In some cases, when the portion of the surface of the fiducial body identifiable as a boundary in a scanned volumetric image comprises one of the feature regions, a unique coordinate mapping between the rigid fiducial body and the scanned volumetric image is determinable from the fiducial plane and the plurality of geometrical characteristics for that feature region. In other cases, when the portion of the surface identifiable as the boundary in the scanned volumetric image comprises at least two of the feature regions, a unique coordinate mapping between the rigid fiducial body and the scanned volumetric image is determinable from the fiducial plane and the plurality of geometrical characteristics for two of the at least two feature regions.

As mentioned above, the appliance can be attached to the human jaw such that the rigid fiducial body is in a fixed spatial relationship with the human jaw. The curved profile of the rigid fiducial body can be shaped to follow a curve of the human jaw. This enables the rigid fiducial body to be positioned close to the human jaw when the appliance is attached thereto. This facilitates scanning the human jaw such that a portion of the rigid fiducial body distinguishable as a boundary in the scanned volumetric image comprises at least one of the feature regions.

The fiducial plane and the geometrical characteristics of at least one feature region of the rigid fiducial body can allow a unique coordinate mapping (e.g. six degrees of freedom) to be determined between the rigid fiducial body and the scanned volumetric image of the human jaw.

In some embodiments, the apparatus may further include a tag that is detachably attachable to the appliance. The tag may include a plurality of trackable markings whose position is trackable by optical, mechanical or electromagnetic pose tracking devices. This may enable a pose of a surgical instrument to be tracked when the appliance is attached to the human jaw and the tag is attached to the appliance.

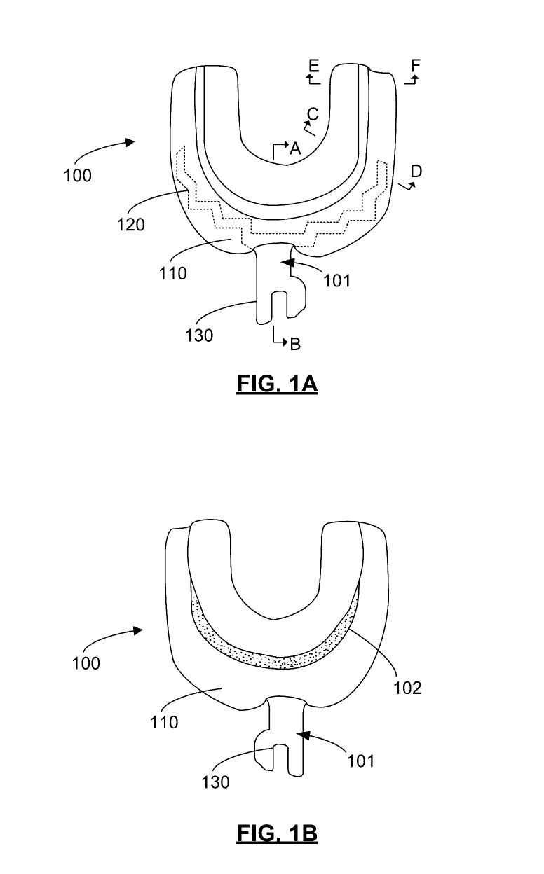

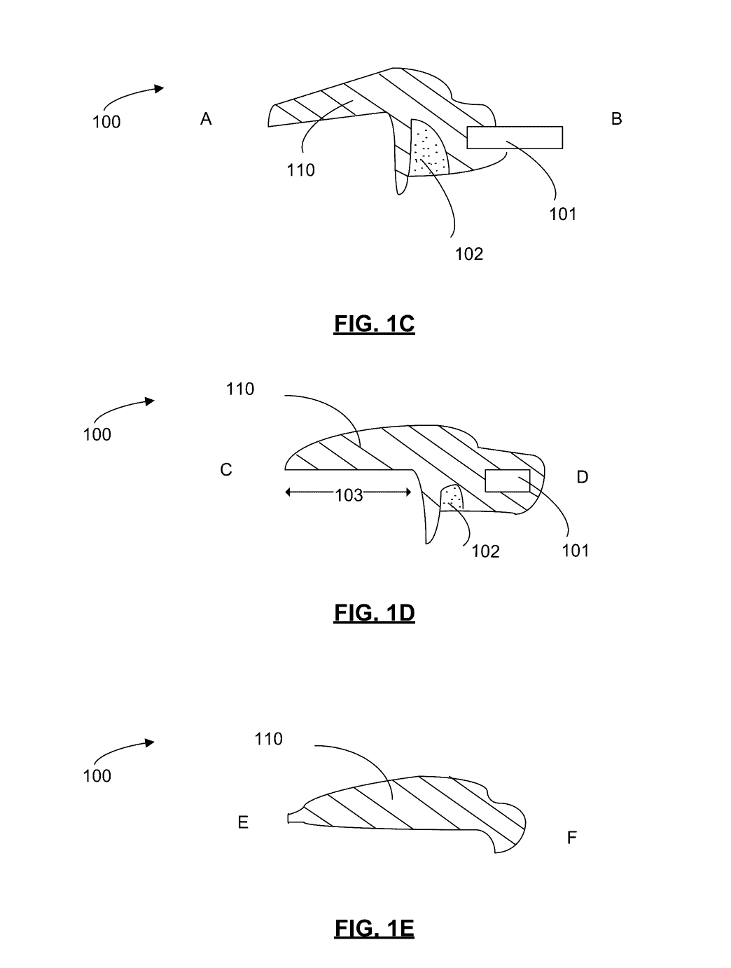

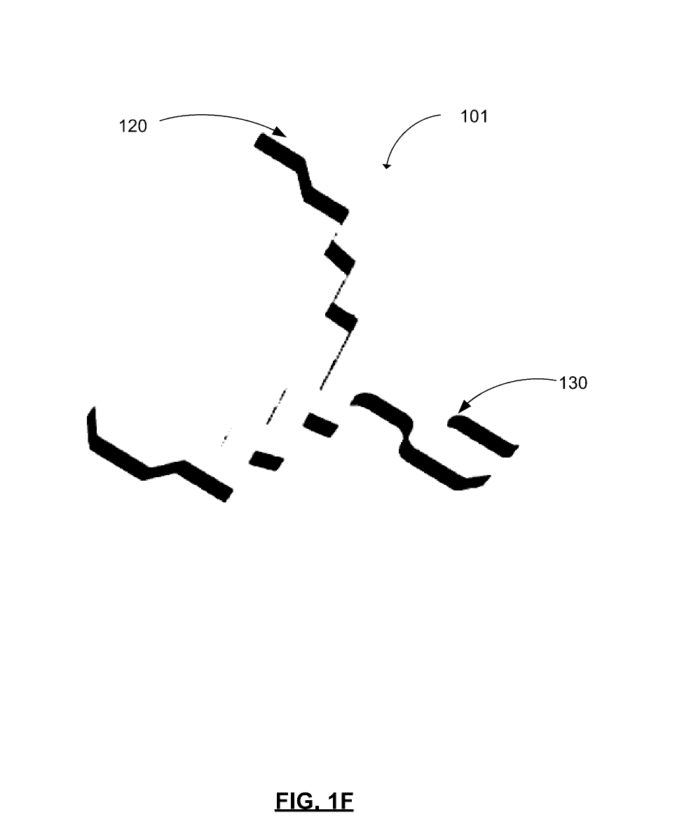

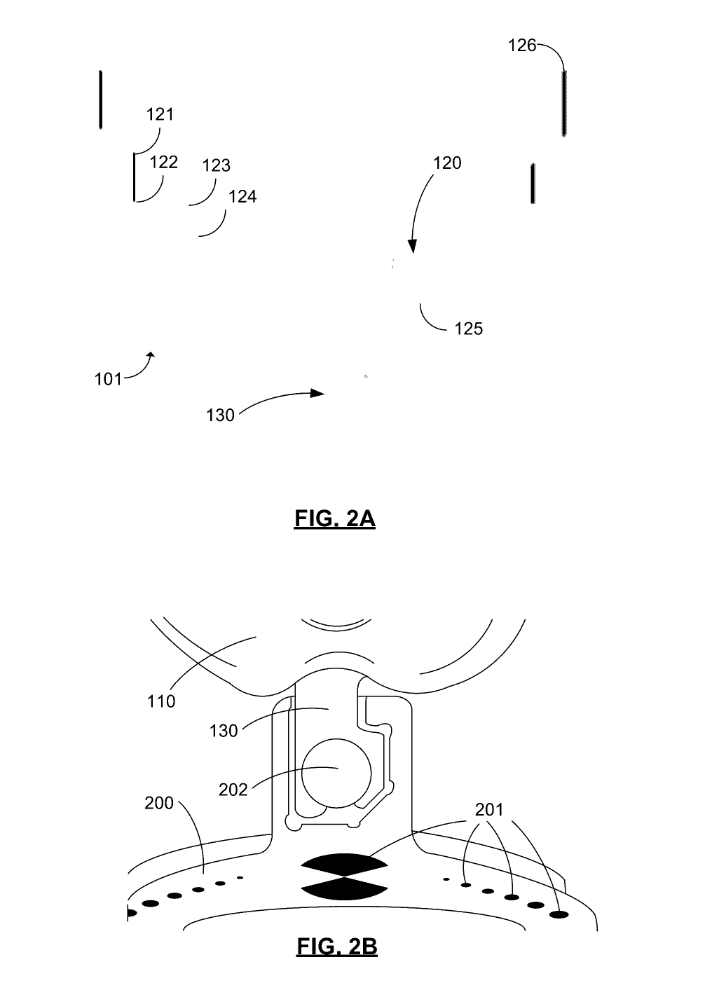

Referring now to FIGS. 1A-1E, shown therein is an example embodiment of an appliance 100 that can be used as a part of an apparatus for registering a human jaw with a scanned volumetric image of the human jaw. In the embodiment shown, the appliance 100 includes a moldable portion 110 (e.g. a retainer sheet), a fiducial 101, and a spacer 102. FIG. 1A shows a view of the side of appliance 100 facing away from the surface of the human jaw to which the appliance 100 may be mated while FIG. 1B shows a view of the surface of appliance 100 that can be shaped to mate with the jaw (i.e. the occlusal side). In some cases, the size of the appliance 100 can be approximately 70×80×8 mm. In the embodiment shown in FIG. 1, the rigid fiducial body 120 is embedded within the moldable portion 110 of the appliance 100.

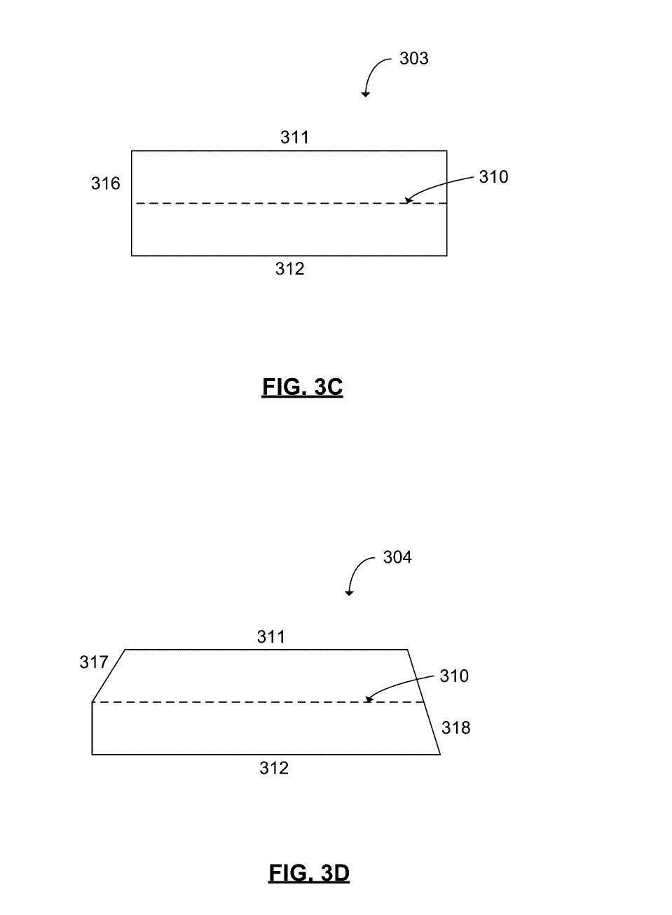

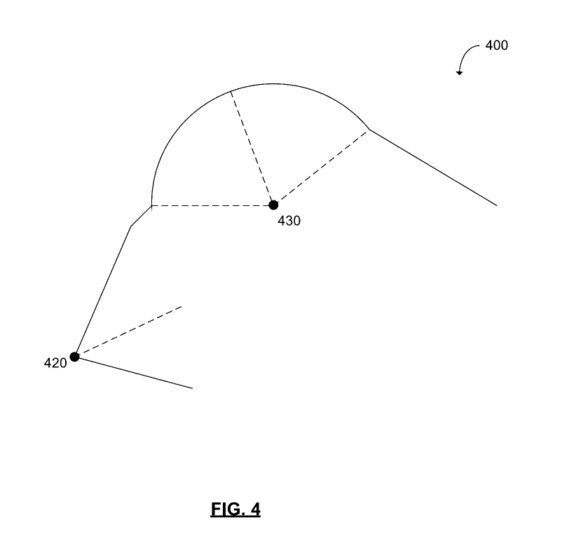

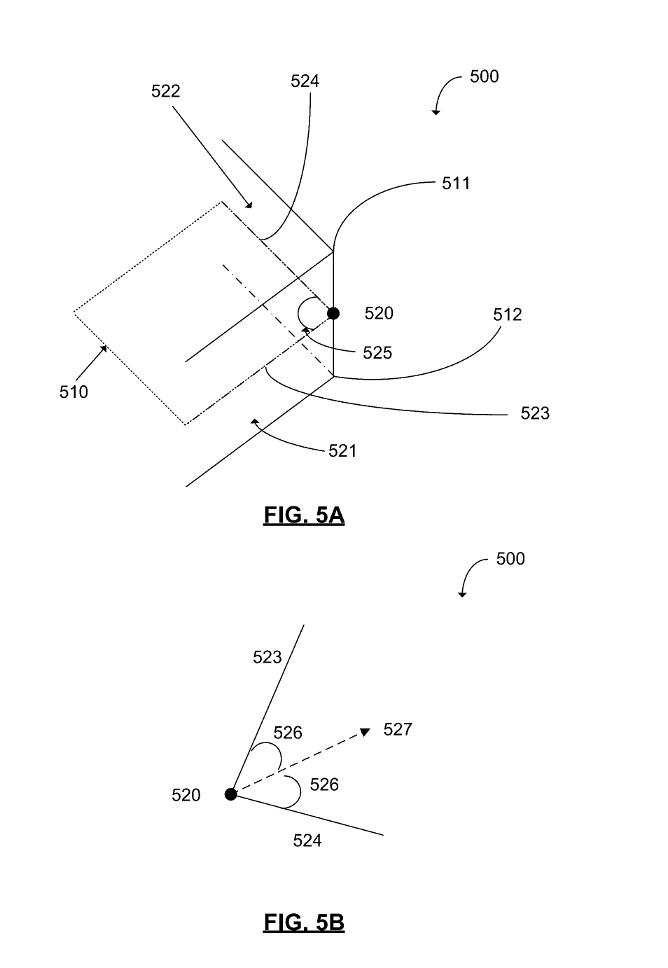

The appliance 100 also includes a tag connection region 130 for indicating a tag connection region, aligning the tag connection region with corresponding surfaces of a tag, and contacting the corresponding surfaces of the tag at the tag connection region such that when the appliance 100 is detachably attached to the tag with the tag connection region 130 in contact with the corresponding surfaces of the tag at the tag connection region the tag is maintainable in a fixed spatial relationship with the rigid fiducial body 120. In the example embodiment shown in FIG. 1, the tag connection region 130 is provided by the fiducial 101. An example embodiment employing the tag connection region 130 will be described in further detail below with reference to FIG. 3.

The rigid fiducial body 120 has a curved profile that is generally shaped to follow a curve of the human jaw. As shown in FIG. 1, the curved profile of the rigid fiducial body 120 is not smooth but includes a number of corner regions along the extent of the rigid fiducial body 120. The appliance 100 may also have a curved apparatus profile that follows a curve of the jaw to allow the apparatus to comfortably mate with a surface of the jaw, such as the teeth.

In some embodiments, the moldable portion 110 can be manufactured of a moldable material which is rigid below a first temperature, the first temperature being selected so that it is rigid at temperatures typically found in the human body. For example, the first temperature can be in a range between 40-50° C., and in some cases it can be below 45° C. The moldable material may become soft and malleable upon heating to a transition temperature below 100° C., but greater than the first temperature. For example, the transition temperature can be such that the moldable portion can be heated to the transition temperature in hot water (e.g. 60-70° C.). For example, the moldable portion 110 can be manufactured from a suitable low temperature thermoplastic material.

The moldable portion 110 can enable the appliance 100 to be moldable to an appliance geometry that mates with a surface geometry of at least a portion of the human jaw, such that when the moldable portion is mated with the human jaw the appliance 100 resists displacement relative to the human jaw. The moldable portion 110 can further be hardenable to remain rigid and resist deformation once molded to the appliance geometry. In some cases, the moldable portion 110 can be hardened upon cooling below a first temperature, the first temperature being lower than the transition temperature.

In some embodiments, a spacer 102 can be included in appliance 100 at a position expected to be near the mesial portion of the jaw when the appliance 100 is attached to the jaw. The spacer 102 can be used to latch the appliance 100 with the buccal side of the front teeth. The spacer 102 may be manufactured of a material configured to be flexible to provide variable spacing for any teeth shape. For example, the spacer 102 can be manufactured from a rubber foam material.

FIGS. 1C, 1D, and 1E show cross-sectional views of the appliance 100 of FIG. 1A along cross-sections A-B, C-D, and E-F, respectively. Cross-sections A-B and C-D are located at a portion of the appliance 100 expected to be near the mesial portion of the jaw. Accordingly, in the example embodiment shown, a spacer 102 can be seen in FIGS. 1C and 1D.

FIGS. 1C and 1D show a portion of the fiducial 101 embedded in the moldable portion 110 of the appliance 100. As mentioned above, in some embodiments a tag connection region 130 can be used to attach a tag to the appliance 100. As shown in FIG. 10, the tag connection region 130 is exposed and extends outwards from the appliance 100.

Cross-section E-F is located at a position of the appliance 100 intended to be positioned near the distal portion of the jaw. In the embodiment shown in FIG. 1, the rigid fiducial body 120 does not extend for the entire length of the appliance 100, and is thus not present in FIG. 1E.

The moldable portion 110 comprises a mating region 103 that can be used for molding the moldable portion 110 to an appliance geometry that mates with the surface geometry of the human jaw. The mating region 103 may have a width that is sufficiently large to conform to a surface geometry of the human jaw. For example, in some cases the mating region 103 can be approximately 12 millimeters.

FIG. 1F shows an example embodiment of a fiducial 101. In some cases, the fiducial 101 may comprise a rigid fiducial body 120 and a tag connection region 130.

The fiducial 101 may be formed such that when the human jaw is scanned with the appliance 100 attached thereto, a representation of at least a portion of the surface of the rigid fiducial body 120 is identifiable as a boundary in the scanned image.

For example, the fiducial 101 can be manufactured from a scan detectable material that provides contrast in scanned images to the moldable portion 110, the human jaw, and air, but does not create image artifacts. For example, in some embodiments the fiducial 101 can be made of aluminum or titanium. This can allow the rigid fiducial body 120 to be identifiable when scanning the human jaw with a scanner such as a CT scanner.

The rigid fiducial body 120 may also have a curved profile shaped to follow a curve of the human jaw. This can ensure that the rigid fiducial body 120 is proximate the human jaw when being scanned. The rigid fiducial body 120 may also include a plurality of feature regions. Each feature region can define a plurality of geometrical characteristics of the rigid fiducial body 120.

The fiducial 101 generally includes a side surface and an opposite side surface spaced apart from the side surface throughout at least the rigid fiducial body 120. At least one of the side surface and the opposite side surface may define a fiducial plane of the rigid fiducial body 120. The distance between the surface and the opposite side surface may define a thickness of the rigid fiducial body 120.

In some cases, such as the example shown in FIG. 1F, the fiducial 101 may be formed as a flat slab. In such cases, the distance between the side surface and the opposite surface may be consistent throughout the rigid fiducial body 120, thus defining a consistent thickness of the rigid fiducial body 120. This can enable the fiducial plane of the rigid fiducial body 120 to be determined from the scanned volumetric image based on knowledge of the thickness of the rigid fiducial body 120.

In some cases where the rigid fiducial body 120 has a consistent thickness, the side surface and the opposite side surface can both be substantially planar and substantially parallel. In such cases, the fiducial plane can be defined as a plane midway between the side surface and the opposite side surface and substantially parallel to both the side surface and the opposite side.

In some cases, the rigid fiducial body 120 can be embedded in the moldable portion 110. During scanning of the human jaw with the appliance 100 attached thereto, a portion of the surface of the rigid fiducial body 120 may be represented in the scanned volumetric image as a boundary. When the representation of the rigid fiducial body 120 in the scanned volumetric image includes at least one feature region, a unique coordinate mapping between the fiducial 101 and the scanned volumetric image may be determined based on the fiducial plane and the geometrical characteristics of the at least one feature region in the scanned volumetric image.

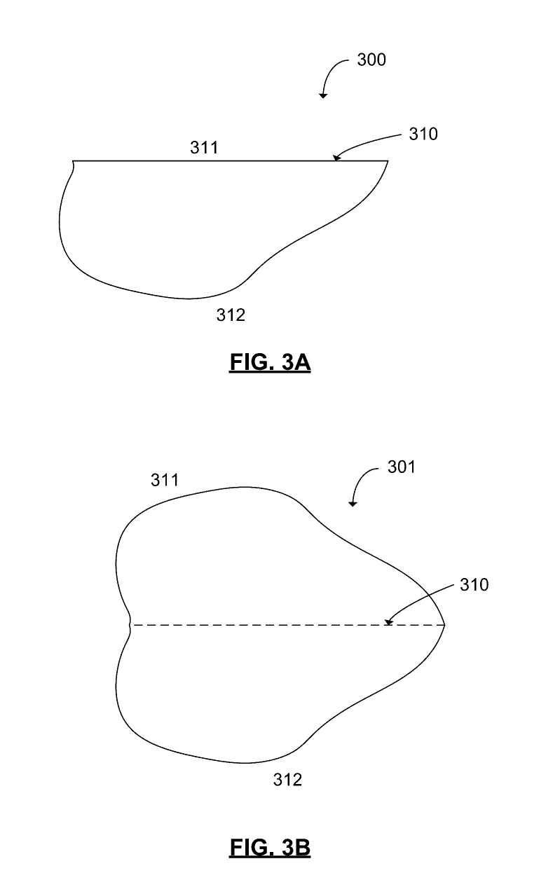

Referring now to FIG. 2A, shown therein is an example embodiment of a fiducial body 101 comprising a rigid fiducial body 120 and a tag connection region 130. The fiducial body 101 shown in FIG. 2A is an example of a flat slab fiducial body, wherein the fiducial plane of a fiducial 101 is parallel to the page and the fiducial 101 has a consistent thickness is in a direction facing inward into the page, or outward from the page. The rigid fiducial body 120 comprises a plurality of corner regions including corner regions 121, 122, 123, 124, 125, and 126. The corner regions 121-126 are examples of feature regions.

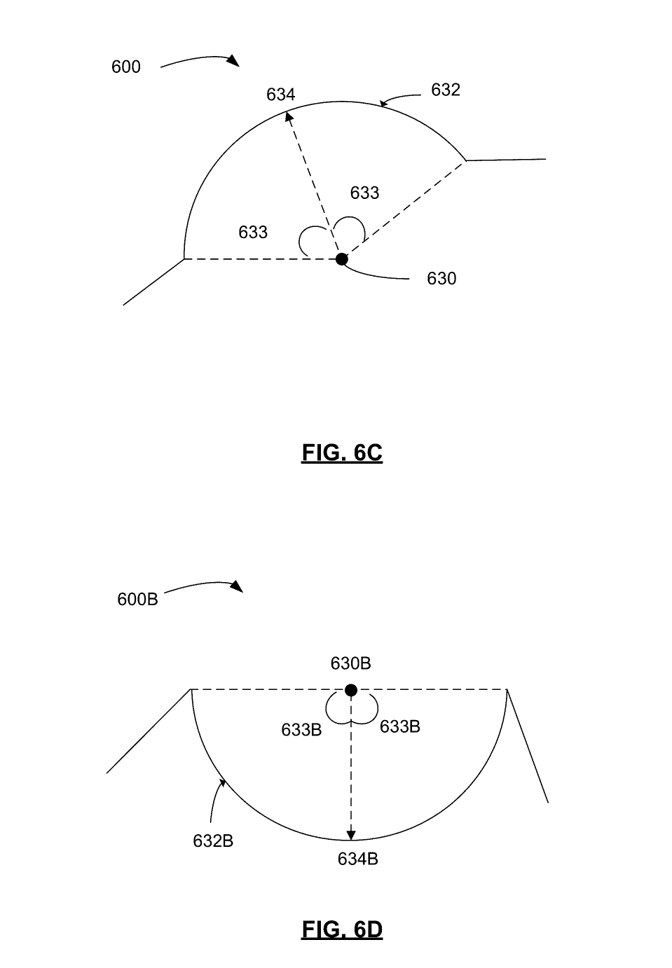

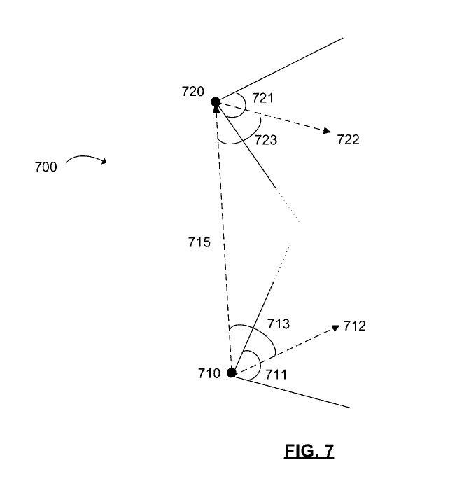

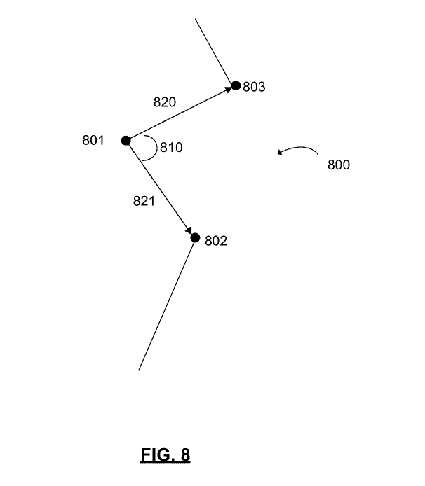

In some embodiments, a unique coordinate mapping between the rigid fiducial body and the scanned volumetric image is determinable from the fiducial plane and the geometrical characteristics of a single corner region (see, for example, embodiments described below with reference to FIG. 3D and FIG. 6). In other embodiments, the geometrical characteristics of two corner regions may be needed to determine the unique coordinate mapping between the rigid fiducial body and the scanned volumetric image (see, for example, embodiments described below with reference to FIGS. 3C, 7 and 8).

Employing a rigid fiducial body 120 with a plurality of feature regions distributed along the entirety of the rigid fiducial body 120 enables a unique coordinate mapping to be determined when only a partial image of the rigid fiducial body 120 is captured during scanning. Various examples of feature regions as well as corresponding geometrical characteristics will be described below with references to FIGS. 4-8.

As mentioned above, the fiducial 101 may be manufactured from a scan detectable material. For example, in some cases where the fiducial 101 is formed as a flat slab, the fiducial 101 may be cut from an aluminum alloy sheet having a thickness of 3 to 5 millimeters.

In some cases, the apparatus may also include a tag 200 that is detachably attachable to the appliance 100. Referring now to FIG. 2B, shown therein is an example embodiment of a tag 200 attached to appliance 100. In the embodiments shown in FIG. 2B, the tag 200 is attached to the appliance 100 by tag connection region 130.

The tag connection region 130 comprises tag engaging surfaces configured to be detachably attached to the tag 200. The tag engaging surfaces indicate the tag connection region 130, align the tag connection region 130 with corresponding surfaces of the tag 200 and contact the corresponding surfaces of the tag at the tag connection region such that when the rigid fiducial body 120 is detachably attached to the tag 200 with the tag engaging surfaces in contact with the corresponding surfaces of the tag 200 at the tag connection region 130 the tag 200 is maintainable in a fixed fiducial spatial relationship with the rigid fiducial body 120. In some cases, the tag 200 may be detachably attached to the appliance 100 using securing mechanisms such as a screw. For example, FIG. 2B shows a thumb screw 202 used to detachably attach the tag 200 to the tag connection region 130.

In some cases, the tag engaging surfaces of the tag connection region 130 may include a tag guide portion, wherein the tag guide portion is configured to guide the corresponding surfaces of the tag 200 into contact with the tag engaging surfaces at the tag connection region 130.

The tag 200 can be constructed to enable it to be tracked by a position tracking system. For example, the tag 200 can include trackable portions, such that when the corresponding surfaces of the tag 200 are aligned and in contact with the tag engaging surfaces at the tag connection region 130 the trackable portions are either optically, electromagnetically or mechanically detectable to determine the positioning and orientation of the corresponding surfaces of the tag 200, such that a pose of the tag engaging surfaces that align with and contact the corresponding surfaces of the tag 200 is determinable and the pose of the rigid fiducial body 120 is determinable. For example, the tag 200 may include a plurality of trackable markings 201. In some cases, the trackable markings may be markings that can be read by a various types of optical tracking devices such as the Micron Tracker by Claronav Inc., for example. Alternatively, the trackable markings 201 may be detectable using mechanical or electromagnetic pose tracking devices.

Referring now to FIGS. 3A-3D, shown therein are cross-sectional views of portions of several example embodiments of a rigid fiducial body.

FIG. 3A shows a cross-section of a first embodiment of a rigid fiducial body portion 300. The rigid fiducial body portion 300 comprises side surface 311 and an opposite side surface 312. The opposite side surface 312 is spaced apart from the side surface 311 throughout the rigid fiducial body. In the rigid fiducial body portion 300, the side surface 311 is substantially planar, such that the side surface 311 defines the fiducial plane 310 of the rigid fiducial body.

As mentioned above, the thickness of the rigid fiducial body can be defined as the distance between the side surface 311 and the opposite side surface 312. In some cases, as shown in rigid fiducial body portion 300 the distance between the side surface 311 and the opposite side surface 312 can vary. Accordingly, in such cases the thickness of the rigid fiducial body can be variable throughout the rigid fiducial body. In such cases, the geometrical characteristics of each of the feature regions in the rigid fiducial body may include the thickness (or a thickness distribution) of the rigid fiducial body in that feature region. In some cases, the thickness of the rigid fiducial body in at least one of the feature regions will be uniform throughout that feature region.

In some cases, for at least some of the feature regions, the thickness (or thickness distribution) of the rigid fiducial body in each one of those feature regions is different from the thickness (or thickness distribution) of the rigid fiducial body in any other feature region. In such cases, when the portion of the surface of the rigid fiducial body identifiable as a boundary in a scanned image includes that feature region, that particular feature region is identifiable based on the thickness (or thickness distribution) of the rigid fiducial body in that feature region.

FIG. 3B shows a cross-section of a second embodiment of a rigid fiducial body portion 301. In FIG. 3B, the rigid fiducial body portion 300 also comprises a side surface 311 and an opposite side surface 312 wherein the opposite side surface 312 is spaced apart from the side surface 311 throughout the rigid fiducial body. In the example shown in FIG. 3B, the side surface 311 and the opposite side surface 312 together define the fiducial plane 310. Furthermore, in this example the side surface 311 and the opposite side surface 312 are substantially symmetric. In some cases, the side surface 311 and the opposite side surface 312 can be asymmetric about the fiducial plane 310 while still together defining the fiducial plane.

FIG. 3C shows a cross-section of a fourth embodiment of a rigid fiducial body portion 303. Rigid fiducial body portion 303 also comprises a side surface 311 and an opposite side surface 312 where the opposite side surface 312 is spaced apart from the side surface 311 throughout the rigid fiducial body. In this example, both the side surface 311 and the opposite side surface 312 are substantially planar throughout the rigid fiducial body. Accordingly, the thickness of the rigid fiducial body is substantially uniform throughout the rigid fiducial body. FIG. 3C is an example embodiment of an apparatus employing a flat-slab fiducial as mentioned above.

In FIG. 3C, the side surface 311 and the opposite side surface 312 together define a fiducial plane 310 and are symmetric about the fiducial plane. The rigid fiducial body portion 303 also includes an edge surface portion 316 extending from the side surface 311 to the opposite side surface 312. In the example shown in rigid fiducial body portion 303, the edge surface portion 316 is substantially orthogonal to the fiducial plane 310 and can be symmetric about the fiducial plane 310 in each feature region.

FIG. 3D shows a cross-section of a fifth embodiment of a rigid fiducial body portion 304. Rigid fiducial body portion 304 also comprises a side surface 311 and an opposite side surface 312 wherein the opposite side surface 312 is spaced apart from the side surface 311 throughout the rigid fiducial body. Furthermore, in rigid fiducial body portion 304 both the side surface 311 and the opposite side surface 312 are substantially planar such that the side surface 311 and the opposite side surface 312 define a fiducial plane 310.

In some cases, the rigid fiducial body may include edge surface portions extending from the side surface 311 to the opposite side surface 312 that are asymmetric. For example, asymmetric edge surface portions can be provided using chamfered or beveled edge portions such as chamfered edge surface portion 317 and beveled edge surface portion 318. In some cases, each feature region in the rigid fiducial body can include an asymmetric edge surface portion extending from the side surface to the opposite side surface. For example, in some cases the various embodiments described herein with reference to FIGS. 4-6 may include asymmetric edge surface portions in each corner region or circle sector region.

Asymmetric edge surface portions enable the unique coordinate mapping between the rigid fiducial body and a scanned image of the rigid fiducial body to be determined based on the fiducial plane and a single corner region or circle sector region. Accordingly, the properties of an asymmetrical edge surface portion may be one of the geometrical characteristics of the feature regions described herein. Within embodiments employing symmetrical edge portions, such as shown in FIG. 3C, even when the identity, location and orientation of a corner region is determinable in the scanned image, it is not possible to determine which of two possible orientations of the fiducial body, twisted 180 degrees around midline 310, is presented. However, such determination can be made when the edge is not symmetrical, as shown in FIG. 3D, and the unique coordinate mapping could be determined based solely on an identification of the fiducial plane and the geometrical characteristics of a single corner region or circle sector region.

Referring now to FIG. 4, shown therein is a top view of a rigid fiducial body portion 400. In FIG. 4, the fiducial plane of rigid fiducial body portion 400 is parallel to the page. Rigid fiducial body portion 400 includes a corner region where the feature point location can be a vertex point 420 defined by the geometrical characteristics of the corner region and the fiducial plane. and a circle sector region where the feature point location can be a center point 430 defined by the geometrical characteristics of the circle sector region and the fiducial plane.

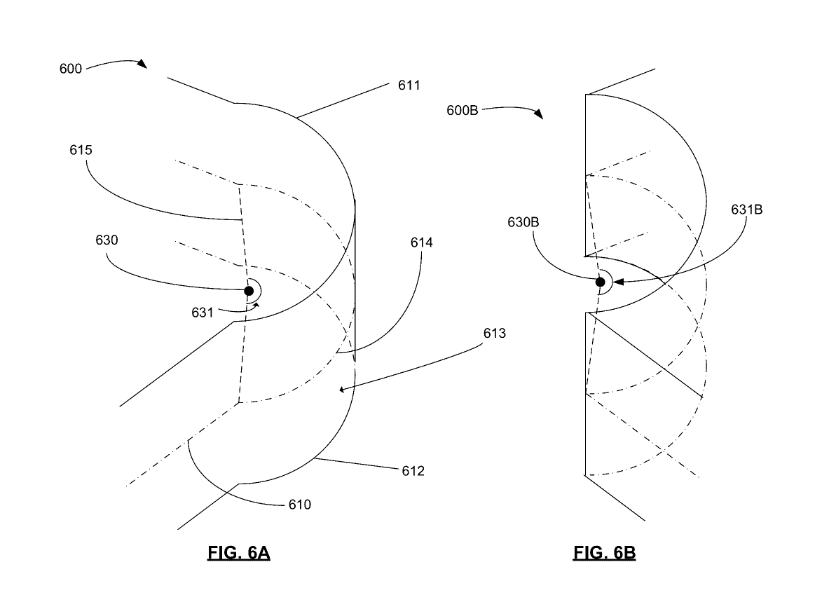

Referring now to FIG. 5A, shown therein is a perspective view of a corner region 500 with vertex point 520 at the fiducial plane 510 shown as the feature point location of corner region 500. The surface of the rigid fiducial body includes a side surface portion and an opposite side surface portion in the corner region 500, which together define a fiducial plane 510. In the example shown in FIG. 5A, the fiducial plane 510 is defined as a plane midway between the side surface portion and the opposite side surface portion that is substantially parallel to both the side surface portion and the opposite side surface portion.

The surface of the rigid fiducial body includes a first edge surface portion 521 extending from the side surface potion to the opposite side surface portion and a second edge surface portion 522 extending from the side surface potion to the opposite side surface portion (the intersection of the first edge surface portion 521, second edge surface portion 522 and side surface portion is shown by 511; the intersection of the first edge surface portion 523, second edge surface portion 521 and opposite side surface portion is shown by 512).

An intersection of the first edge surface portion 521 and the fiducial plane 510 within the corner region 500 defines a first edge intersecting line 523 that is substantially straight. An intersection of the second edge surface portion 522 and the fiducial plane 510 within the corner region 500 defines a second edge intersecting line 524 that is substantially straight. A vertex point 520 (i.e. the feature point location) can be formed by an intersection of the first edge intersecting line 523 and the second edge intersecting line 524 at the fiducial plane 510. The vertex point 520 (i.e. the feature point location) can also be considered an intersection between the fiducial plane 510 and an intersection line defined by the intersection of the first edge surface portion 521 and the second edge surface portion 522. Alternatively, the fiducial plane may be defined by a side surface, in which case the feature point would be located at a corner 511 or 512.

The vertex point 520 has an opening angle 525 formed by the intersection of the first edge intersecting line 523 and the second edge intersecting line 524 at the vertex point 520. The opening angle of a vertex point is generally defined by an angle along the interior of the rigid fiducial body and may be acute, obtuse, reflex, or right angles. The geometrical characteristics of the corner region 500 can include the magnitude of the opening angle. In some cases, for each feature region where the feature point location is a vertex point, the opening angle magnitude can be distinct from the opening angle magnitude of every other feature region including a vertex point.

Referring now to FIG. 5B, shown therein is a top view of the corner region 500. The corner region 500 may define an opening angle bisecting direction 527 that originates from the vertex point 520 and bisects the opening angle into two equal angles 527. The geometrical characteristic of the corner region 500 may also include the opening angle bisecting direction 527.

In at least one embodiment, the opening angle magnitude of each corner region where the feature point location is a vertex point differs from the opening angle magnitude of every other corner region comprising a vertex point. In such an embodiment, each particular corner region can be uniquely identified in a scanned image of the rigid fiducial body by identifying an opening angle magnitude of a corner region representation in the scanned image. In other embodiments, the opening angle magnitude can be considered together with other geometrical characteristics of the corner region, for example the thickness of the rigid fiducial body in that corner region and/or the approximate orientation of the angle bisecting direction relative to the patient, to uniquely identify each corner region. Furthermore, in such embodiments where a corner region further includes an asymmetrical edge portion, such as a beveled or chamfered edge, the unique coordinate mapping between the rigid fiducial body and the scanned image of the rigid fiducial body can be determined by identifying the fiducial plane and the geometrical characteristics of that corner region alone.

Referring now to FIG. 6A shown therein is a perspective view of a circle sector region 600 of the rigid fiducial body where the feature point location is a center point 630 at the fiducial plane 610. The rigid fiducial body comprises a side surface portion and an opposite side surface portion which together define a fiducial plane 610. A first edge surface portion 613 extends from the side surface portion to the opposite side surface portion (611 shows the intersection of the first edge surface portion 613 and the side surface portion, while 612 shows the intersection of the first edge surface portion 613 and the opposite side surface portion) in the center region.

The first edge surface portion 613 has a circular segment profile such that a projection of the circular segment profile onto the fiducial plane 610 is a circular segment 614. The circular segment 614 has a circle segment radius 615. The center point 630 can be defined by the intersection of the fiducial plane and the center of the circular segment. The geometrical characteristics of the circle sector region 600 include the circle segment radius 615. In circle sector 600, the circular segment profile of the first edge surface portion 613 can define a central axis. The feature point location of the circle sector region 600 can be defined as the intersection between the central axis and the fiducial plane 610, which in this case is center point 630. The fiducial plane may be defined differently, for example by one of the flat sides (top or bottom side in FIGS. 6A and 6B), in which case the intersection with the fiducial plane defining the feature point will lie elsewhere along the central axis.

In some embodiments, for each feature region where the feature point location is a center point, the circle segment radius of that feature region can be different from the circle segment radius of any other feature region including a center point. In such an embodiment, when the portion of the surface of the rigid fiducial body that is identifiable as a boundary in a scanned volumetric image includes at least a portion of the first edge surface portion, that feature region can be identifiable based on the circle segment radius, and the circle segment radius can be identifiable from the portion of the first edge surface portion in the scanned image.

The center point 630 of circle sector region 600 has an opening angle 631 defined by an angle subtended by the first edge surface portion through the center point. The geometrical characteristics of the circle sector region 600 may include the opening angle magnitude of the opening angle 631. In some embodiments, for each circle sector region where the feature point location is a center point, the opening angle magnitude for that feature region can be distinct from the opening angle magnitude of every other feature region that includes a center point. In such embodiments, each circle sector region can be uniquely identified based on the opening angle magnitude of that circle sector region. In other embodiments, the opening angle magnitude can be considered together with other geometrical characteristics of the circle sector region, for example the thickness of the rigid fiducial body in that region and/or the circle segment radius, to uniquely identify each corner region.

In some embodiments, where each circle sector region is uniquely identifiable on the basis of the geometrical characteristics of that circle sector region alone (i.e. each circle sector region has at least one of a unique circle segment radius and an unique opening angle magnitude), and a circle sector region includes at least one edge portion that is asymmetrical about the fiducial plane (the geometrical characteristics of that circle sector region thereby including the properties of the asymmetrical edge portion), the unique coordinate mapping between the rigid fiducial body and a scanned image of the rigid fiducial body can be uniquely determined based on the fiducial plane and the geometrical characteristics as well as the feature point location for that circle sector region alone.

Referring now to FIG. 6B, shown therein is an example embodiment of a circle sector region 600B. In some embodiments, the feature point location (center point) of a circle sector region may be located on the interior of the rigid fiducial body or the feature point location (center point) may not be located on the interior of the rigid fiducial body. Circle sector region 600B is an example of a circle sector region where the center point 630B is located outside of the rigid fiducial body.

Referring now to FIGS. 6C and 6D, shown therein are top views of the projections 632 and 632B of the circular segment profile of the circle sector regions 600 and 600B respectively on to a fiducial plane. As shown in FIG. 6C, the circle sector region 600 defines an opening angle bisecting direction 634 in a direction originating from the center point 630 and bisecting the opening angle 631 into two equal angles 633. In some cases, the geometrical characteristic of a circle sector region may include the opening angle bisecting direction. Similarly, the circle sector region 600B also defines an opening angle 631B about the center point 630B and an opening angle bisecting direction 634B bisecting the opening angle 631B into two equal angles 633B.

In some embodiments, the combination of geometric characteristics of a single feature region may be insufficient to uniquely determine the mapping between the scanned image and the fiducial coordinates. For example, there may be multiple corner regions with the same geometrical characteristics and/or some corner regions may have rotational symmetry. In such embodiments, pairs of feature regions can provide a unique mapping when geometrical characteristics of the feature region pairs, such as the distance between feature point locations and additional cross-region characteristics are considered as well. Such feature region pair geometrical characteristics may include, for example, the angle formed between each of the two angle bi-sector vectors and a line connecting the two feature point locations. In such embodiments, each feature region pair includes a first feature region and a second feature region, where each region pair has a distinct combination of geometrical characteristics. In such embodiments, any first feature region can be distinguished from any one of the other feature regions based on the combination of geometrical characteristics for any feature region pair including the first feature region.

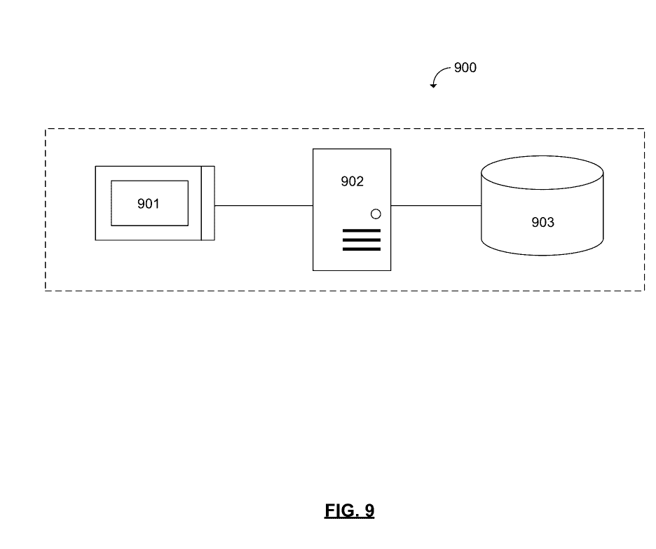

Referring now to FIG. 7, shown therein is an example embodiment of a feature regions pair 700 including a first feature region 710 and a second feature region 720. Although feature regions pair 700 is shown including feature regions (710 and 720) that are corner regions having vertex points as the feature point locations, in other embodiments, feature regions employing circle segment regions with center points can be used. In other embodiments, feature regions pairs including a combination of a corner region having a vertex point and a circle segment region having a center point can also be used.

As shown in FIG. 7, the first feature region 710 and the second feature region 720 of the regions pair 700 do not need to be immediately adjacent on the surface of the rigid fiducial body. That is, in some cases the portion of the surface identifiable as a boundary may include the first feature region and the second feature regions in disjoint portions in the scanned volumetric image.

For example, in some cases the portion of the surface of the rigid fiducial body identifiable as a boundary in the scanned volumetric image may include a first sub-portion comprising the first feature region and a second sub-portion comprising the second feature region, where the first sub-portion and the second sub-portion are disjoint, or indeed separated by a unscanned portion of the rigid fiducial body, in the scanned volumetric image. Nonetheless, in such embodiments the first feature region and the second feature region may still form a feature region pair.

The first feature region 710 has an opening angle 711 with a corresponding opening angle magnitude and an opening angle bisecting vector 712. Similarly, the second feature region 720 has an opening angle 721 with a corresponding opening angle magnitude and an opening angle bisecting vector 722. The feature region pair 700 defines a first pair vector 715 from the feature point location of the first feature region 710 to the feature point location of the second feature region 720.

The combination of geometrical characteristics for the feature region pair 700 may include: a first opening angle magnitude, the first opening angle magnitude being the magnitude of the opening angle 711 of the first feature region 710; a second opening angle magnitude, the second opening angle magnitude being the magnitude of the opening angle 721 of the second feature region 720; the magnitude of the first pair vector 715; a first vector bisector angle 713 defined by an angle between the first pair vector 715 and an opening angle bisecting vector 712 in a direction originating from the feature point location of the first feature region 710 and bisecting the opening angle 711 of the first feature region 710; and a second vector bisector angle 723 defined by an angle between the first pair vector 715 and an opening angle bisecting vector 722 in a direction originating from the feature point location of the second feature region 720 and bisecting the opening angle 721 of the second feature region 720.

Referring now to FIG. 8 shown therein is an example embodiment of a feature region 800 comprising a first feature region 801, a first adjacent feature region 802, and a second adjacent feature region 803.

The geometrical characteristics of the feature region 800 can include the opening angle magnitude 810 of the first feature region 801, the distance 821 from the feature point location of the first feature region 801 to the feature point location of the first adjacent feature region 802, and the distance 822 from the feature point location of the first feature region 801 to the feature point location of the second adjacent feature region 803. In some cases, each feature region can have a unique combination of the opening angle magnitude of that feature region, the distance from that feature region to the first adjacent feature region, and the distance from that feature region to the second adjacent feature region. The embodiment shown in FIG. 2A (discussed above) is one such example.

In some cases, a first feature region and a second feature region may each have the same opening angle magnitude, but the distance from the feature point location of the first feature region to one of its adjacent feature regions would then be different from the distance from the second feature region to one of its adjacent feature regions.

To prevent the false identification of feature regions where the accuracy of scanned images is less than ideal, in some cases the rigid fiducial body can be designed such that the geometrical characteristics of the feature regions have a corresponding range such that no two geometrical characteristics fall within the same range. For example, in FIG. 2A, vertex point 123 may have an opening angle magnitude of 105°, a distance of 4 millimeters to vertex point 124, and a distance of 5.5 millimeters to adjacent vertex point 122. The rigid fiducial body 120 can be designed so that no other corners have a combination of features that includes an opening angle magnitude of 105±2° and a distance of 4.0±0.2 millimeters to a first adjacent feature region and a distance of 5.5±0.2 millimeters to second adjacent feature region. Thus, when vertex point 123 appears in a scanned volumetric image, an estimation of its opening angle magnitude and the distances to two adjacent feature regions would provide a reliable identification of the vertex point, even when the estimate is not entirely accurate.

In embodiments of the system and apparatus described herein, each feature region may be identifiable when the scanned volumetric image includes a small portion of the rigid fiducial body comprising the feature region. For example, vertex point 123 may be identified by a region as small as 10 millimeters in diameter by positioning vertex point 123 at the center of the region. This can be particularly advantageous when the patient is scanned by a dental CT scanner with a limited field of view, e.g., with less than 80 mm diameter.

In some cases, when additional feature regions are included in the scanned volumetric image they can be used to improve the accuracy of the coordinate mapping but are not required to determine the coordinate mapping. Including a large number of feature regions on the rigid fiducial body generally increases the robustness of the registration process by enabling it to succeed even when only a small portion of the rigid fiducial body is detected in the scanned volumetric image.

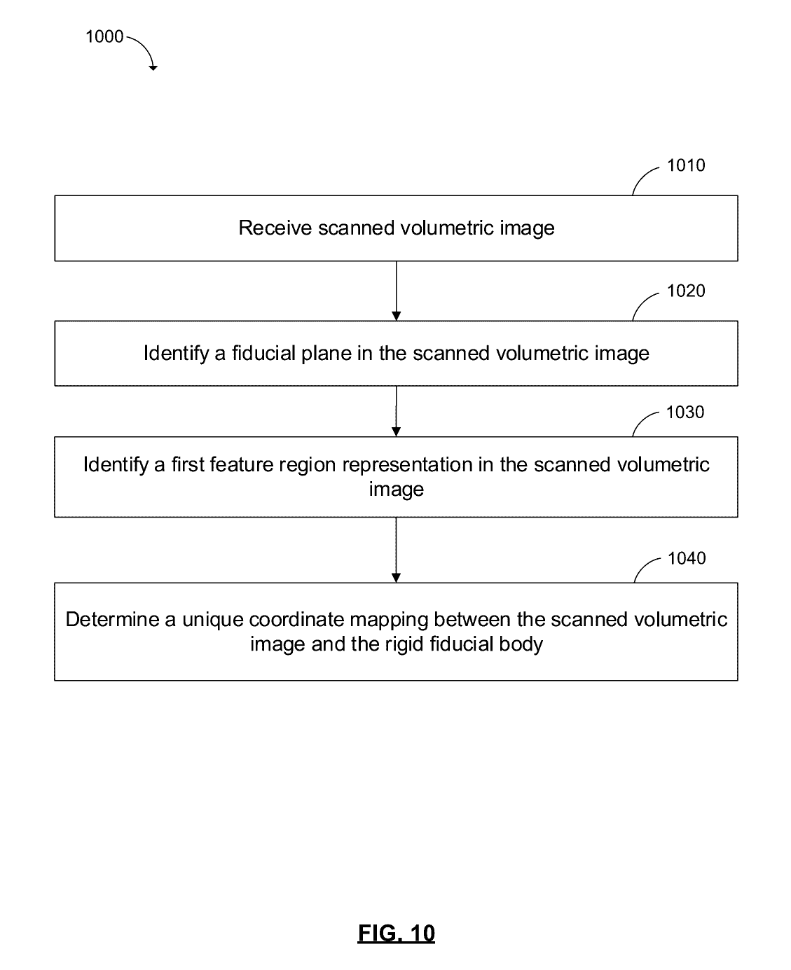

Referring now to FIG. 9, shown therein is an example embodiment of a system 900 for registering a human jaw with a scanned volumetric image of the human jaw. The system 900 may include a scanner 901, a data processor 902, and a database memory 903.

The scanner 901 can be configured for conducting a scan of the human jaw with an appliance attached thereto. The appliance can include any of the various embodiments of an appliance comprising a rigid fiducial body described herein. The scanner 901 can further be configured to provide a representation of the human jaw and the portion of a surface of the rigid fiducial body distinguishable as the boundary in the scanned volumetric image, the portion of the rigid fiducial body comprising at least one feature region, each feature region defining having a plurality of geometrical characteristics.

In some embodiments, the scanner 901 can be any suitable scanner for acquiring a volumetric tomography image of an anatomical region and a rigid fiducial body proximate the anatomical region. For example, the scanner 901 can be a tomographical scanner such as a CT (computed tomography) scanner.

The data processor 902 can be coupled to the scanner 901 and the database memory 903. In some cases, the data processor 902 may be located remotely from the scanner 901 and/or the database memory 903, while in other cases the components of the system 900 may be coupled locally. Generally, the data processor 902 can be configured to identify a fiducial plane and the plurality of geometrical characteristics corresponding to at least one feature region in a scanned image.

The data processor 902 can be configured to receive a scanned volumetric image from the scanner 901. The data processor 902 can then be configured to identify a fiducial plane in the scanned volumetric image and identify a first feature region representation in the scanned volumetric image based on a plurality of geometric characteristics of the first feature region representation. The first feature region representation may correspond to a first feature region in the portion of the surface of the rigid fiducial body. The data processor 902 can also be configured to determine a unique coordinate mapping between the scanned volumetric image and the rigid fiducial body using the identified fiducial plane and the geometrical characteristics of the identified first feature region.

In some cases, the system 900 also includes a database memory 903. The database memory 903 can be configured to store a reference model of the rigid fiducial body, comprising the locations and geometrical characteristics of feature regions in the reference fiducial coordinate system.

In some embodiments employing a database memory 903, the data processor 902 can be operated to map the coordinate space of the scanned volumetric image received from scanner 901 by obtaining a mapping transformation from the reference model of the fiducial 101 to the scanned volumetric image based on identifying in the scanned volumetric image the fiducial plane and at least one feature region representation.

In some cases, the system 900 may also include the appliance, where the appliance is repeatably attachable to the human jaw. The appliance may include a rigid fiducial body where a surface of the rigid fiducial body comprises a plurality of feature regions and each feature region defines a plurality of geometrical characteristics of the rigid fiducial body. The surface of the rigid fiducial body can also include a side surface and an opposite side surface spaced apart from the side surface throughout the rigid fiducial body where at least one of the side surface and the opposite side surface define the fiducial plane. In some cases, the surface of the rigid fiducial body is shaped to maintain a substantially uniform distance from a portion of the external surface of an average human jaw. In various embodiments, the system 900 may include any of the embodiments of an appliance described herein.

Referring now to FIG. 10, shown therein is a flowchart of an example method 1000 for determining a unique coordinate mapping between a scanned volumetric image and a rigid fiducial body. Method 1000 is an example of a method that can be performed by data processor 902.

At 1010, data processor 902 receives a scanned volumetric image from the scanner 901. In some cases, the scanned volumetric image can be received from the database memory 903, for example when the volumetric image is obtained by the scanner 901 and stored for later processing by the data processor 902. The scanned volumetric image received by the data processor 902 generally includes a portion of a surface of the rigid fiducial body distinguishable as the boundary in the scanned volumetric image. In some cases, the portion of the surface of the rigid fiducial body comprises at least one feature region, each feature region having a plurality of geometrical characteristics. In other cases, the portion of the surface of the rigid fiducial body comprises at least two feature regions.

At 1020, the data processor 902 may identify a fiducial plane in the scanned volumetric image. For example, the fiducial plane may be identified based on geometrical characteristics determined from the portion of the surface of the rigid fiducial body included in the scanned volumetric image. Many image processing techniques are known for identifying a flat planar region in a volumetric image, for example using edge detection followed by a Hough transform. For example, pairs of flat planar regions at the expected distance from each other can be matched to identify portions of the rigid fiducial body and compute the fiducial plane.

The fiducial plane of the rigid fiducial body generally corresponds to one of three planes of the coordinate space of the fiducial. The fiducial plane may be defined by a side surface and an opposing side surface of the surface of the rigid fiducial body in any of the ways described herein above. In some cases, for example those employing a flat-slab fiducial (see e.g. FIGS. 3C, 3D, 5A, 6A and 6B above) by locating a flat-slab region (i.e. a region of consistent thickness) in the scanned volumetric image and further identifying a central plane of this flat-slab region, the first of three planes of the coordinate space of the scanned volumetric image may be determined.

At 1030, a first feature region representation and a first feature point location representation may be identified in the scanned volumetric image. The first feature region representation may be identified based on a plurality of geometric characteristics of the first feature region representation and the fiducial plane in the scanned volumetric image. For example, this can be done by computing a reformatted (resampled) slice along the fiducial plane and applying a 2D feature detector to the image, for example a corner detector to detect a corner and a circle detector to detect a section of a circle. Many algorithms are known in the image processing field for finding such features. In some cases, a second feature region representation and a second feature point location representation may also be identified. This may occur for example, where the system is used with an appliance that has feature region pairs or otherwise may require two feature regions to be identified to determine the unique coordinate mapping. In some such cases, each of the first feature region representation and the second feature region representation may be identified based on geometrical characteristics of the pair of feature region representations including those two region representations.

At 1040, the unique coordinate mapping between the scanned volumetric image and the rigid fiducial body can then be determined by the data processor 902. For example, in some cases the data processor 902 may identify a reference fiducial plane in a stored reference model of the rigid fiducial body. The data processor 902 may also identified a first reference feature region in the stored reference model corresponding to the identified first feature region representation.

The first reference feature region corresponding to the first feature region representation may be identified as a reference feature region having geometrical characteristics that correspond to the geometrical characteristics of the first feature region representation. For example, as mentioned above, when each feature region of the rigid fiducial body has a distinct opening angle magnitude or a distinct circle segment radius, the first reference feature region can be identified as the reference feature region having an opening angle magnitude or distinct circle segment radius that is substantially identical to, or within a known range, of the first feature region representation. In some cases, a second reference feature region may also be identified.

Once a reference fiducial plane and a first reference feature region corresponding to the first feature region representation are identified the data processor 902 may align the representation of the rigid fiducial body with the stored reference model by aligning the identified fiducial plane with the reference fiducial plane and aligning the geometrical characteristics of the identified first reference feature region representation and the feature point location of the first reference feature region with corresponding geometrical characteristics of the identified first feature region representation and the first feature point location representation to obtain a mapping transformation.

In some cases, the reference fiducial plane may first be aligned with the identified fiducial plane of the scanned volumetric image. The remaining mapping transformation may be obtained by a two-dimensional registration process of the portion of the surface of the rigid fiducial body in the scanned volumetric image. The remaining two planes of the coordinate space of the scanned volumetric image may relate to the location and orientation of the rigid fiducial body disposed along the first plane.

Once a mapping transformation between the coordinate spaces of the reference model of the fiducial body and the representation of the fiducial body in the scanned volumetric image is obtained, this transformation can be used to map the human jaw to its appearance in the scanned image, since the relationship between the fiducial body and the jaw is substantially identical in both coordinate spaces. This mapping transformation together with a measured pose of the jaw can then be used to map locations in the interior of the human jaw to corresponding locations in the scanned volumetric image of the interior of the human jaw. A data processor 902 may be operated to compute and track the pose of the jaw by applying various pose tracking methods, such as those described in PCT Application No. PCT/CA2011/001294.

In some cases, after scanning, the appliance 100 may be removed from the human jaw. Subsequently, a tag 200 may be releasably attached to the appliance 100 to provide a pose tracking apparatus. The tag 200 may be detachably attached to tag engaging surfaces such that when the tag is attached to the tag engaging surfaces, the tag is maintainable in a fixed spatial relationship with the rigid fiducial body. The pose tracking apparatus can then be attached to the human jaw prior to surgery wherein pose tracking will be used.

The tag 200 may be automatically mapped to the coordinate space of the scanned volumetric image without additional steps to determine the geometrical relationship between the tag 200 and the fiducial 101. This mapping transformation may be pre-defined by the tag connection region of the appliance 100 used to detachably attach the tag 200 to the appliance 100. The tag engaging surfaces provided by the appliance 100 allow the spatial relationship between the rigid fiducial body and the position of the tag 200 on the apparatus to be determined in advance.

As the geometrical relationship between the rigid fiducial body and the tag 200 does not depend on the molding of the moldable portion 110, the tag 200 can generally be used with a plurality of different appliances 100 customized for different patients.

Various embodiments have been described herein by way of example only. Various modification and variations may be made to these example embodiments without departing from the spirit and scope of the invention, which is limited only by the appended claims.

1. An apparatus for registering a human jaw with a scanned volumetric image of the human jaw, the apparatus comprising:
an appliance repeatably attachable to the human jaw, the appliance comprising a rigid fiducial body and constructed such that when the appliance is attached to the human jaw the rigid fiducial body is in a fixed spatial relationship with the human jaw;
wherein:
a surface of the rigid fiducial body comprises a side surface and an opposite side surface, the opposite side surface spaced apart from the side surface throughout the rigid fiducial body, at least one of the side surface and the opposite side surface defining a fiducial plane;
the surface of the rigid fiducial body further comprises a plurality of feature regions, each feature region defining a feature point location and a plurality of geometrical characteristics associated with that feature point location;
the rigid fiducial body has a curved profile such that a projection of the curved profile onto the fiducial plane is generally curved, the curved profile being shaped to follow a curve of the human jaw when the appliance is attached to the human jaw;
the rigid fiducial body is formed such that when the human jaw is tomographically scanned with the appliance attached thereto, a representation of at least a portion of the surface of the rigid fiducial body is identifiable as a boundary in the scanned volumetric image;
when the portion of the surface identifiable as the boundary in the scanned volumetric image comprises two of the feature regions, a unique coordinate mapping between the rigid fiducial body and the scanned volumetric image is determinable from the fiducial plane and the plurality of geometrical characteristics for each of the two feature regions; and,
the plurality of geometrical characteristics for each of the two feature regions and the fiducial plane are determinable from the boundary in the scanned volumetric image.

2. An apparatus for registering a human jaw with a scanned volumetric image of the human jaw, the apparatus comprising:
an appliance repeatably attachable to the human jaw, the appliance comprising a rigid fiducial body and constructed such that when the appliance is attached to the human jaw the rigid fiducial body is in a fixed spatial relationship with the human jaw;
wherein:
a surface of the rigid fiducial body comprises a plurality of feature regions, each feature region defining a feature point location and a plurality of geometrical characteristics associated with that feature point location;
the surface of the rigid fiducial body further comprises a side surface and an opposite side surface, the opposite side surface spaced apart from the side surface throughout the rigid fiducial body, at least one of the side surface and the opposite side surface defining a fiducial plane;
the rigid fiducial body has a curved profile such that a projection of the curved profile onto the fiducial plane is generally curved, the curved profile being shaped to follow a curve of the human jaw when the appliance is attached to the human jaw;
the rigid fiducial body is formed such that when the human jaw is tomographically scanned with the appliance attached thereto, a representation of at least a portion of the surface of the rigid fiducial body is identifiable as a boundary in the scanned volumetric image;
when the portion of the surface identifiable as the boundary in the scanned volumetric image comprises one of the feature regions, a unique coordinate mapping between the rigid fiducial body and the scanned volumetric image is determinable from the fiducial plane and the plurality of geometrical characteristics for that feature region; and,
the fiducial plane and the plurality of geometrical characteristics of that feature region are determinable from the boundary in the scanned volumetric image.

3. The apparatus according to claim 2, wherein the side surface and the opposite side surface together define the fiducial plane and are substantially symmetrical about the fiducial plane.

4. The apparatus according to claim 2, wherein the side surface is substantially planar such that the side surface defines the fiducial plane.

5. The apparatus according to claim 2, wherein the curved profile is substantially arch shaped.

6. The apparatus according to claim 2, wherein the curved profile is shaped to keep a substantially uniform distance between the surface of the rigid fiducial body and a portion of an external surface of an average human jaw.

7. The apparatus according to claim 2, wherein the curved profile is shaped to keep a substantially uniform distance between the surface of the rigid fiducial body and a portion of a buccal surface of an average human jaw.

8. The apparatus according to claim 2, wherein a distance between the side surface and the opposite side surface defines a thickness of the rigid fiducial body; and
for a first feature region of the plurality of feature regions, the thickness of the rigid fiducial body in that first feature region is different from the thickness of the rigid fiducial body in a second feature region, the thickness of the rigid fiducial body in the first feature region is one of the geometrical characteristics of the first feature region, and the thickness of the rigid fiducial body in the second feature region is one of the geometrical characteristics of the second feature region.

9. The apparatus according to claim 2, wherein the feature point location of each feature region is one of a center point and a vertex point.

10. The apparatus according to claim 9, wherein when the feature point location of at least one of the feature regions is a vertex point, and for each feature region where the feature point location is a vertex point:
the surface of the rigid fiducial body comprises a first edge surface portion extending from the side surface to the opposite side surface in that feature region, and a second edge surface portion extending from the side surface to the opposite side surface in that feature region;
an intersection of the first edge surface portion and the fiducial plane within the feature region defines a first edge intersecting line that is substantially straight, an intersection of the second edge surface portion and the fiducial plane within the feature region defines a second edge intersecting line that is substantially straight;
the vertex point is formed by an intersection of the first edge intersecting line and the second edge intersecting line at the fiducial plane;
the vertex point has an opening angle formed by the intersection of the first edge intersecting line and the second edge intersecting line at the vertex point, the opening angle having an opening angle magnitude; and
the geometrical characteristics of that feature region comprise the opening angle magnitude.

11. The apparatus according to claim 10, wherein for a first feature region where the feature point location is a vertex point, the opening angle magnitude is distinct from the opening angle magnitude of a second feature region where the feature point location is a vertex point, the opening angle magnitude of the first feature region is one of the geometrical characteristics of the first feature region and the opening angle magnitude of the second feature region is one of the geometrical characteristics of the second feature region.

12. The apparatus according to claim 10, wherein for each feature region where the feature point location is a vertex point:
that feature region defines an opening angle bisecting direction in a direction originating from the vertex point and bisecting the opening angle; and
the geometrical characteristics of that feature region comprise the opening angle bisecting direction.

13. The apparatus according to claim 9, wherein the feature point location of at least one of the feature regions is a center point, and for each feature region where the feature point location is a center point:
the surface of the rigid fiducial body comprises a first edge surface portion extending from the side surface to the opposite side surface in that feature region, the first edge surface portion having a circular segment profile such that a projection of the circular segment profile onto the fiducial plane is a circular segment, the circular segment profile having a circle segment radius;
an intersection of the fiducial plane and a center of the circular segment defines the center point; and
the geometrical characteristics of that feature region comprise the circle segment radius.

14. The apparatus according to claim 13, wherein for each feature region where the feature point location is a center point:
the circle segment radius of that feature region is different from the circle segment radius of any other feature region comprising a center point.

15. The apparatus according to claim 13, wherein for each feature region where the feature point location is a center point:
the center point has an opening angle defined by an angle subtended by the first edge surface portion through the center point, the opening angle having an opening angle magnitude; and
the geometrical characteristics of that feature region comprise the opening angle magnitude.

16. The apparatus according to claim 13, wherein for each feature region where the feature point location is a center point:
a combination of the circle segment radius and the opening angle magnitude of that feature region is different from a combination of circle segment radius and opening angle magnitude of any other feature region comprising a center point.

17. The apparatus according to claim 15, wherein for each feature region where the feature point location is a center point:
the feature region defines an opening angle bisecting direction in a direction originating from the center point and bisecting the opening angle; and
the geometrical characteristics of that feature region comprise the opening angle bisecting direction.

18. The apparatus according to claim 2, wherein for each feature region the surface of the rigid fiducial body comprises at least one edge surface portion extending from the side surface to the opposite side surface that is asymmetrical about the fiducial plane within that feature region.

19. The apparatus according to claim 1, wherein the rigid fiducial body further comprises:
a plurality of feature region pairs, each feature region pair including a first feature region and a second feature region, each feature region pair having a distinct combination of geometrical characteristics; and
when the portion of the surface identifiable as the boundary in the scanned volumetric image comprises any feature region pair, the unique coordinate mapping between the rigid fiducial body and the scanned volumetric image is determinable from the fiducial plane and the combination of geometrical characteristics for that feature region pair.

20. The apparatus according to claim 19, wherein the portion of the surface identifiable as the boundary in the scanned volumetric image comprises a first sub-portion comprising the first feature region of the feature region pair and a second sub-portion comprising the second feature region of the feature region pair; and
the first sub-portion and the second sub-portion are disjoint in the scanned volumetric image.

21. The apparatus according to claim 19, wherein the combination of geometrical characteristics for any feature region pair comprises:
a first opening angle magnitude of an opening angle of the first feature region;
a second opening angle magnitude of an opening angle of the second feature region;
a magnitude of a first pair vector from a feature point location of the first feature region to a feature point location of the second feature region;
a first vector bisector angle defined by an angle between the first pair vector and an opening angle bisecting vector in a direction originating from the feature point location of the first feature region and bisecting the opening angle of the first feature region; and
a second vector bisector angle defined by an angle between the first pair vector and an opening angle bisecting vector in a direction originating from the feature point location of the second feature region and bisecting the opening angle of the second feature region.

22. The apparatus according to claim 1, wherein:
the geometrical characteristics of each feature region include a unique combination of an opening angle magnitude of an opening angle of that feature region, a distance from a feature point location of that feature region to a feature point location of a first adjacent feature region, and a distance from the feature point location of that feature region to a feature point location of a second adjacent feature region.

23. The apparatus according to claim 2 wherein the appliance is configured to be moldable to an appliance geometry that mates with a surface geometry of at least a portion of the human jaw, such that when mated with the human jaw, the appliance resists displacement relative to the human jaw, the appliance being further configured to be hardenable to remain rigid and resist deformation once molded to the appliance geometry.

24. The apparatus according to claim 23, wherein a portion of the appliance is rigid below 45° C., the portion becomes moldable when heated to a transition temperature greater than 45° C. and less than 100° C., the portion being subsequently hardenable when cooled below 45° C.

25. A system for registering a human jaw with a scanned volumetric image of the human jaw, the system comprising:
a tomographic scanner for conducting a scan of the human jaw with an apparatus attached thereto, the apparatus comprising a rigid fiducial body and constructed such that the rigid fiducial body is maintained in a fixed spatial relationship with the human jaw when the apparatus is attached thereto, the rigid fiducial body is formed such that when the human jaw is scanned with the apparatus attached thereto, a representation of at least a portion of the surface of the rigid fiducial body is identifiable as a boundary in the scanned volumetric image, the scanner being configured to provide a representation of the human jaw and the portion of a surface of the rigid fiducial body distinguishable as the boundary in the scanned volumetric image, the portion of the rigid fiducial body comprising at least one feature region, each feature region having a plurality of geometrical characteristics; and
a data processor coupled to the tomographic scanner, the data processor being configured to:
receive the scanned volumetric image from the tomographic scanner;
identify a fiducial plane in the scanned volumetric image;
identify a first feature region representation and a first feature point location representation in the scanned volumetric image based on a plurality of geometric characteristics of the first feature region representation and the fiducial plane, the first feature region representation corresponding to a first feature region in the portion of the surface of the rigid fiducial body; and
determine a unique coordinate mapping between the scanned volumetric image and the rigid fiducial body using the identified fiducial plane and the geometrical characteristics of the identified first feature region.
